(19) United States
(12) Patent Application Publication
(10) Pub. No.: US 2019/0209709 A1
(43) Pub. Date: Jul. 11, 2019

(54) SYSTEM AND METHOD TO MODULATE PAIN AND ITCH THROUGH CUTANEOUS TRANSFER OF GENETIC INFORMATION

(71) Applicant: Circuit Therapeutics, Inc., Menlo Park, CA (US)

(72) Inventors: Christopher L. Towne, San Francisco, CA (US); Dan Andersen, Menlo Park, CA (US)

(73) Assignee: Circuit Therapeutics, Inc., Menlo Park, CA (US)

(21) Appl. No.: 16/361,181

(22) Filed: Mar. 21, 2019

Related U.S. Application Data
(63) Continuation of application No. 15/428,095, filed on Feb. 8, 2017.
(60) Provisional application No. 62/292,771, filed on Feb. 8, 2016.
(60) Provisional application No. 62/320,422, filed on Apr. 8, 2016.
(60) Provisional application No. 62/418,758, filed on Nov. 7, 2016.

(51) Int. Cl. A61K 48/00; A61N 5/06; C12N 7/00; A61K 38/16; C12N 15/87; A61K 9/00; C12N 15/113; A61K 41/00; A61K 35/76

(52) U.S. Cl. CPC A61K 48/0066; A61N 2005/067; C12N 7/00; A61N 5/06; A61K 38/16; A61N 5/0616; C12N 15/87; A61K 9/0019; C12N 15/1138; A61N 5/062; A61K 48/0075; A61K 41/00; A61K 35/76; A61N 2005/0664; A61N 5/0622

(57) ABSTRACT

One embodiment is directed to a system for altering the function of a sensory unit that innervates a targeted tissue region in an animal, the sensory unit being configured to express a light-responsive protein, comprising a light delivery element configured to direct radiation to at least a portion of a targeted tissue structure; and a light source configured to provide light to the light delivery element; wherein the targeted tissue structure is illuminated transcutaneously with radiation such that a membrane potential of cells comprising the targeted tissue structure is modulated at least in part due to exposure of the light-responsive protein to the radiation.

RELATED APPLICATION DATA

The present application is a continuation of U.S. patent application Ser. No. 15/428,095, filed on Feb. 8, 2017, which claims priority to U.S. Provisional Application Ser. No. 62/292,771, filed Feb. 8, 2016, U.S. Provisional Application Ser. No. 62/320,422, filed Apr. 8, 2016 and to U.S. Provisional Application Ser. No. 62/418,758, filed Nov. 7, 2016. The foregoing applications are hereby incorporated by reference into the present application in their entirety.

### INCORPORATION-BY-REFERENCE OF MATERIAL SUBMITTED ELECTRONICALLY

Incorporated by reference in its entirety is a computer-readable nucleotide/amino acid sequence listing submitted concurrently herewith, and identified as follows: One 14.7 KiloByte ASCII (Text) file named “20053_SeqList_ST25.txt” created on Mar. 21, 2019.

### FIELD OF THE INVENTION

The present invention relates generally to systems, devices, and processes for facilitating various levels of control over cells and tissues in vivo, and more particularly to systems and methods for therapeutic modulation of pain and itch through cutaneous transfer of genetic information.

### BACKGROUND

The sensation of pain and itch arise following the activation of nerve endings of primary sensory neurons in the dermis and epidermis of skin. Electrical signals (action potentials) are generated at the nerve endings and propagate along the axon where they are delivered to the central nervous system at the spinal cord or brainstem. The cell bodies of primary sensory neurons are located in dorsal root ganglia (“DRG”) or trigeminal ganglia (“TG”) and contain the DNA that codes for the proteins that are expressed along the entire length of the neuron.

Ion channels and pumps maintain the membrane voltage across neurons and facilitate the generation and propagation of action potentials. Some approved pharmacological agents for treating pain and itch function by decreasing the electrical excitability of primary sensory neurons by inhibiting these ion channels. For example, lidocaine blocks voltage-gated sodium (Na+) ion channels and morphine activates opioid receptors that reduce voltage-gated calcium (Ca21) ion channel activity. It is notable that while agents such as lidocaine and morphine are relatively powerful analgesics, they are not specific in their impact systemically, targeting many voltage-gated ion channels in nontargeted parts of the body, such as in the heart, and central locations, which limits their therapeutic utility; further, such agents have been found to be related to severe addition problems in the United States and elsewhere. It is well accepted that reducing electrical excitability of primary sensory neurons can inhibit pain.

Gene therapy is the therapeutic delivery of genetic information (nucleic acid polymers, such as DNA and RNA) into a patient's cells to treat disease. The genetic information can be delivered via viral or non-viral methods and can encode the expression of transgenic protein or information to reduce levels of a patient's existing protein.

Gene therapy methods have been used to deliver genes to primary sensory neurons in animal models of pain. Transgenic proteins have been expressed to reduce the electrical excitability of primary sensory neurons. For example, expression of the endogenous opioid peptide enkephalin, which is the natural ligand for the delta opioid receptor, can reduce pain levels in animal models. Optogenetic proteins, that utilize microbial light-sensitive, or light-responsive, ion transporters (channels and pumps), have also been expressed to modulate electrical excitability and reduce pain. Some of these optogenetic paradigms involve the delivery of light transdermally or intradermally to activate the ion channel or pump to reduce electrical excitability and block transmission of pain signals. Delivery of genetic information to reduce levels of an endogenous target protein have also been utilized in animal models to reduce pain. For example, expression of small hairpin RNA (“shRNA”) against voltage-gated sodium channels have reduced electrical excitability and inhibited pain in animal models.

Gene therapy using these methods may have several benefits compared with traditional pharmaceuticals in the treatment of pain and itch. Therapeutic genetic information can be delivered to specific regions and/or cells in the neuraxis, resulting in a localized concentration of the genetic information, reducing systemic off target effects, and also mitigating at least some of the aforementioned drug-related downsides.

Delivery of genetic information to the skin is attractive because it allows targeting to the specific region affected by pain or itch. Here, the therapeutic genetic information may be taken up by cutaneous cells themselves, or by the primary sensory nerve endings in the dermal or epidermal layers to result in reduction of pain or itch at the desired location.

### SUMMARY

One embodiment is directed to a method for altering the function of the sensory unit that innervates a targeted tissue region in a mammal comprising the steps of identifying the targeted tissue region; cutaneously administering into the targeted tissue region an adeno-associated virus wherein the viral genome encodes at least one exogenous protein; expressing the exogenous protein in the targeted sensory unit; and altering the function of the targeted sensory unit to treat or restore the sensory response because of the exogenous protein expression while not impacting the function of nearby sensory units. The adeno associated virus may have a coat protein selected from the group consisting of adeno-associated virus strain 1, adeno-associated virus strain 6, and adeno-associated virus strain 8. The exogenous protein may be a light-responsive protein and expressing the exogenous protein in the targeted sensory unit further may comprise exposing the targeted sensory unit to light. The light-responsive protein may be a stimulatory opsin. The stimulatory opsin may be selected from the group consisting of ChR2, C1V1-T, C1V1-TT, CatCh, VChR1-SFO, and ChR2-SFO. The light-responsive protein may be an inhibitory opsin. The inhibitory opsin may be selected from the group consisting of NpHR, eNpHR 1.0, eNpHR 2.0, eNpHR 3.0, Mac, Mac 3.0, Arch, ArchT, iChR, iC1C2, iC++, SwiChR++, and JAWS. The exogenous protein may be one which reduces pain by decreasing electrical excitability, or by modulating receptors, neurotransmitters, ion channels, second messenger systems, and biochemical mediators of inflammation that underlie pain. The exogenous protein may be selected from the group consisting of P2X, DOR, Nav 1.7, Nav 1.8, Cav 1.2, NR2B, mACHR subtype M2, mAChR subtype M3, mAChR subtype M4, NTS2, Homer1, Shank1, TRPV1, DREAM, CCR2, GDNF, NR2B, PKCγ, Toll-like receptor 4, NR1 subunit of NMDA, connexin 43, GABA, endomorphin, and a ligand associated G-protein. The targeted tissue region may be selected based at least in part upon an undesired sensory response selected from the group consisting of acute pain, chronic pain, allodynia, ectopic pain, neuropathic pain, itch, and parathesia. The targeted tissue region may be selected based at least in part upon anesthesia. The targeted tissue region may be selected based at least in part upon a feeling of satiation. The adeno-associated virus may be self-complementary. Cutaneously administering may comprise intradermally or subcutaneously administering.

Another embodiment is directed to a method of treating or preventing an undesired or lack of sensory response of a region of tissue by altering the function of the sensory unit that innervates that tissue region in a mammal comprising the steps of identifying the tissue region that has, will have, or is lacking the sensory response; cutaneously administering into the identified tissue region an adeno-associated virus comprising a coat protein selected from the group consisting of adeno-associated virus strain 1, adeno-associated virus strain 6, and adeno-associated virus strain 8 where the viral genome encodes at least one molecule that results in RNAi; expressing the RNAi molecule in the targeted sensory unit; and altering the function of the targeted sensory unit to treat or restore the sensory response because of the RNAi expression while not impacting the function of nearby sensory units. In an embodiment wherein the undesired sensory response is pain, the RNAi may be specific to reducing the expression of a protein selected from the group consisting of P2X, DOR, Nav 1.7, Nav 1.8, Cav 1.2, NR2B, mACHR subtype M2, mAChR subtype M3, mAChR subtype M4, NTS2, Homer1, Shank1, TRPV1, DREAM, CCR2, GDNF, NR2B, PKCγ, Toll-like receptor 4, NR1 subunit of NMDA, and connexin 43. The undesired sensory response may be selected from the group consisting of acute pain, chronic pain, allodynia, ectopic pain, neuropathic pain, itch, and parathesia. The lack of sensory response may be anesthesia. The lack of sensory response may be a feeling of satiation. The undesired sensory response may be chronic pain and the RNAi may be achieved through a ddRNAi specific to Nav 1.7. The adeno-associated virus may be self-complementary. Cutaneously administering comprises intradermally or subcutaneously administering.

Another embodiment is directed to a method of treating neuropathic pain in a region of tissue by altering the function of the sensory unit that innervates that tissue region in a mammal comprising the steps of identifying the tissue region that has the undesired neuropathic pain; cutaneously administering into the identified tissue region an adeno-associated virus comprising a strain 6 coat protein where the genome encodes the opsin iC++; expressing iC++ in the targeted sensory unit and exposing the sensory unit to light; and reducing the neuropathic pain in the tissue region innervated by the sensory unit while not impacting the function of nearby sensory units. The adeno-associated virus is self-complementary. Cutaneously administering may comprise intradermally or subcutaneously administering.

Another embodiment is directed to a method of treating superficial somatic pain in a region of tissue by altering the function of the sensory unit that innervates that tissue region in a mammal comprising the steps of identifying the tissue region that has the undesired superficial somatic pain; cutaneously administering into the identified tissue region an adeno-associated virus comprising a strain 6 coat protein where the genome that encodes iC++; expressing iC++ in the targeted sensory unit and exposing the sensory unit to light; and reducing the superficial somatic pain in the tissue region innervated by the sensory unit while not impacting the function of nearby sensory units. The AAV is self-complementary. Cutaneously administering may comprise intradermally or subcutaneously administering.

Another embodiment is directed to a system for altering the function of a sensory unit that innervates a targeted tissue region in an animal, the sensory unit being configured to express a light-responsive protein, comprising a light delivery element configured to direct radiation to at least a portion of a targeted tissue structure; and a light source configured to provide light to the light delivery element; wherein the targeted tissue structure is illuminated transcutaneously with radiation such that a membrane potential of cells comprising the targeted tissue structure is modulated at least in part due to exposure of the light-responsive protein to the radiation. The light source may be selected from the group consisting of a laser, a light emitting diode, and a chemiluminescent compound. The sensory unit may be adjacent a stratum corneum layer that has been altered prior to administration of one or more clinical compounds configured to cause the sensory unit to express the light-responsive protein. The stratum corneum layer may be altered using a configuration selected from the group consisting of: a tape stripping configuration, a dermabrasion configuration, a microdermabrasion configuration, a depilatory compound application configuration, a sonophoresis configuration, an iontophoresis configuration, an electroporation configuration, a microdermabrasion configuration, a microneedle configuration, a laser ablation configuration, and an optoporation configuration. The light-responsive protein may be a stimulatory opsin. The stimulatory opsin may be selected from the group consisting of ChR2, C1V1-T, C1V1-TT, CatCh, VChR1-SFO, and ChR2-SFO. The light-responsive protein may be an inhibitory opsin. The inhibitory opsin may be selected from the group consisting of NpHR, eNpHR 1.0, eNpHR 2.0, eNpHR 3.0, Mac, Mac 3.0, Arch, ArchT, iChR, iC1C2, iC++, SwiChR++, and JAWS. The sensory unit may be configured to express the light-responsive protein via administration into the targeted tissue region of an adeno-associated virus wherein a viral genome encodes at least one light responsive protein which becomes expressed in the sensory unit. The adeno associated virus may have a coat protein selected from the group consisting of adeno-associated virus strain 1, adeno-associated virus strain 6, and adeno-associated virus strain 8. The targeted tissue region may be selected based at least in part upon an undesired sensory response selected from the group consisting of acute pain, chronic pain, allodynia, ectopic pain, neuropathic pain, itch, and parathesia. The targeted tissue region may be selected based at least in part upon anesthesia. The targeted tissue region may be selected based at least in part upon a feeling of satiation. The adeno-associated virus may be self-complementary. The light source may be a chemiluminescent compound created using a chemiluminescent reaction that is based at least in part upon a peroxyoxalate oxidation reaction.

### BRIEF DESCRIPTION OF THE DRAWINGS

FIG. 1 illustrates aspects of one embodiment wherein an AAV strain carrying the IC++ opsin is administered into the hind paw of a mouse using subcutaneous injection.

FIG. 2 illustrates a chart featuring mechanical threshold data pertinent to one embodiment, wherein in the presence of light, pain is inhibited (increased mechanical threshold levels are shown) following subcutaneous injection with AAV expressing iC++, relative to the injection of the vehicle (saline) alone.

FIG. 3A illustrates a sequence alignment of five different opsins including iC++. The bolded first 11 amino acids indicate a sequence change between C1C2 and iC++ (the addition of the first 11 amino acids of channelrhodopsin-2, for improved membrane trafficking). The bolded amino acids in iC1C2, iC1C2++and SwiChR++ are mutations from C1C2. The bolded amino acids in GtACR2 are key residues for ion selectivity.

FIG. 3B illustrates a vector structure between the two inverted terminal repeats (Left-ITR and Right-ITR) of the AAV strains discussed in the illustrative embodiments below. iC++ is the inhibitory opsin, hSyn is the human synapsin 1 promoter and p2a is an expression tag. WPRE is the woodchuck hepatitis post-transcriptional regulatory element and pA is a poly-A signaling element.

FIG. 4A illustrates initial steps of one embodiment of an experimental incision pain model.

FIG. 4B illustrates a testing phase of one embodiment of an experimental incision pain model.

FIG. 4C illustrates change in withdrawal threshold in one embodiment (increase=treatment) before, during, and after the incision phase of a pain model with vehicle only (saline) injection. Dark-shaded bars represent a “light-on” condition, while light-shaded bars represent a “light-off” condition. Withdrawal threshold is plotted in terms of grams (g) on the vertical axis, with days before, during, and after incision on the horizontal axis.

FIG. 4D illustrates change in withdrawal threshold with injection of an AAV1 encoding iC++. Starred bars are statistically significant in the depicted experimental data.

FIG. 4E illustrates change in withdrawal threshold with injection of an AAV5 encoding iC++.

FIG. 4F illustrates change in withdrawal threshold with injection of an AAV6 encoding iC++. Starred bars are statistically significant.

FIG. 4G illustrates change in withdrawal threshold with injection of an AAV8 encoding iC++. Starred bars are statistically significant.

Various AAV strains carrying the IC++ opsin may be administered into the hind paw of the mouse using subcutaneous injection. Results from such an experimental configuration are illustrated in FIGS. 4A-4G and FIG. 5.

FIG. 5 illustrates the percent of neurons expressing iC++ as indicated by histology. Expression is categorized into high, mid, and low based on staining intensity.

FIG. 6A illustrates the first two steps of the incision pain model utilized in the experiments of FIG. 6, done in rats.

FIG. 6B illustrates the testing phase of the incision pain model utilized in the experiments of FIG. 6, done in rats.

FIG. 6C illustrates the change in withdrawal threshold (increase=treatment) at pre-virus administration (light off only), before, during and after the incision phase of the pain model with vehicle only (saline) injection. Dark-shaded bars represent a “light on” condition and light-shaded bars represent “light off”. Withdrawal threshold is plotted in terms of grams (g) on the y-axis with days before, during, and after incision in the x-axis. Single and double starred bars are statistically significant.

FIG. 6D illustrates the change in withdrawal threshold (increase=treatment) at pre-virus administration (“light off” condition only), before, during, and after the incision phase of the pain model with AAV6 encoding iC++ injection. Dark-shaded bars represent a “light on” condition and light-shaded bars represent “light off”. Withdrawal threshold is plotted in terms of grams (g) on the y-axis with days before, during, and after incision in the x-axis. Single and double stared bars are statistically significant.

FIG. 7A illustrates the first two steps of the chronic constriction pain model utilized in the experiments of FIG. 7, done in rats.

FIG. 7B illustrates the testing phase of the chronic constriction pain model utilized in the experiments of FIG. 7, done in rats.

FIG. 7C graphs the change in withdrawal threshold (increase=treatment) before, during and after the nerve constriction and injection with vehicle only (saline) or AAV6: iC++. Light-shaded bars represent a “light off” condition, dark-shaded bars represent “blue light on”, and hatched bars are “yellow light on”. Withdrawal threshold is plotted in terms of grams (g) on the y-axis with days before, during, and after incision in the x-axis.

FIG. 7D illustrates the percent neurons expressed compared to change in withdrawal threshold for the animals of the experiment of FIGS. 7A-C.

FIG. 8A illustrates the experimental design to illustrate the on- and off-target effect of injection of the virus in the lateral paw as compared to the medial paw in a mouse.

FIG. 8B illustrates the increase in withdrawal threshold on the medial sensory unit and the lateral sensory unit with blue light on (dark shaded bar) and light off (light shaded bar). Starred bars are statistically significant, “n.s.” is not statistically significant.

FIG. 9A illustrates the administration of AAV and fast blue dye in both intradermal injection and nerve injection.

FIG. 9B illustrates the percent of neurons expressing iC++ with nerve compared to intradermal injections.

FIG. 9C illustrates the percent of iC++ expressing neurons that stain with fast blue with intradermal and nerve injection.

FIG. 10 illustrates the comparative percent GFP expression with back injection and hindpaw injection administration of AAV6 in parent and self-complementing form.

FIGS. 11 through 13 illustrate exemplary system level deployment of an optogenetic treatment system for intervention in accordance with the present invention.

### DETAILED DESCRIPTION

Presented herein are systems, methods, and configurations for modulating pain and itch through cutaneous, such as intradermal or subcutaneous, transfer of genetic information.

In various embodiments, the subject methods and configurations provide for targeted alteration of the sensory response of one sensory unit. A sensory unit is that area of tissue plus those particular nerve or nerves that carry sensory information within that tissue where the nerve endings terminate in an area of the skin of a certain area. The present specification provides a means of delivering genetic information in a targeted manner through a virus or other delivery method such that it will result in transduction of a limited number of nerve cells, specifically, those of a sensory unit. When using a virus used for transduction it carries within its genome an endogenous gene or other genetic information. The resulting transduction of the sensory nerves by the virus, and the expression of the endogenous gene or genetic information, alters the sensory response of the tissue region and in that region only. In this way, the response of the targeted sensory unit is altered while not impacting the function of nearby sensory units.

This is a surprising result for several reasons. First, it was assumed in the art that the level of transduction obtained by a virus observed through skin administration was below the threshold that would be necessary for effective change through viral transduction and expression of genetic information in the function of a sensory unit. The data provided herein shows that this is not the case. Second, it was not known whether skin directed administration would result in selective transduction—that is, neighboring sensory units would be not impacted by such an administration method. The data provided herein shows that this is the case. The skin directed administration described, whether transdermally or subcutaneous, does result in the desired targeted transduction of a sensory unit, resulting in the desired change of function in the targeted sensory unit without altering the function of nearby, but non-targeted sensory units. In this way, the present method provides a method of selectively altering a sensory response through transdermal or subcutaneous administration of the virus into the sensory unit whose function is to be changed.

In various embodiments, this approach may be taken to enable optogenetic treatment methods, systems, or configurations. In one embodiment, genetic information encoding a light-activated ion transporter (such as the blue-light activated chloride ion channel, iC++) is delivered to the epidermis and/or dermis by adeno-associated virus (AAV) and taken up by the primary sensory nerve endings. This genetic information is transported to the cell body in the dorsal root ganglion (DRG) or trigeminal ganglion (TG) to result in expression of the light-activated ion channel protein, that is trafficked back down to the primary sensory nerve endings in skin, where they may be modulated by intradermal light delivery to reduce pain. Light may also be delivered at the level of the particular nerve, at the level of the DRG, or the spinal cord, such as by the use of implantable light delivery technologies. Other examples of light-activated ion transporters include but are not limited to the blue light excitatory opsin, channelrhodopsin2 (ChR2 and variants), the inhibitory yellow light-activated chloride pump, halorhodopsin (NpHR and variants), the blue-green light driven proton pump Mac (and variants), the green light-activated proton pump, Arch (and variants), and the red light-activated halorphdopsin JAWS (and variants). Reference is made to Chow et al. Nature, 463:98-102 (2010); Chuong et al. Nature Neuroscience 17:1123-1129 (2014); Berdnt et al. Proc. Natl Acad Sci USA, vol. 113(4): 822-829 (2016), and patent application publication US20130347137, each of which is incorporated by reference herein in its entirety. Step function opsins such as ChR2(C128A) or ChR2(1285) can also be used a stimulatory opsin or SwiChR++ as an inhibitory opsin.

In each of these optogenetic embodiments, the light-activated ion transporter is utilized to alter the function of the sensory unit through the altered, commonly increased, transport of the ion. As indicated by the variety of opsins that can be utilized, this approach can be used to either excite or inhibit the production of action potentials within the neurons of a particular sensory unit with the delivery of light. The result is dependent on the identity and direction of the ion transport provided by the opsin. In particular, if the ion is positively charged and the movement is into the cell or if the ion is negatively charged and the movement is out of the cell, increased transport results in a stimulation of the cell expressing the opsin (e.g. an increased chance of an action potential, or depolarization). This is generally known as “stimulation.”

Conversely, if the ion is negatively charged and the movement is into the cell or the ion is positively charged and the movement is out of the cell, increased transport results in an inhibition of the cell expressing the opsin (e.g. a decreased chance of an action potential, or hyperpolarization). This is generally known as “inhibition.” A possible specific approach is the use of these optogenetic methods to treat pain, both acute and chronic.

In another embodiment, this approach is taken to enable a protein therapeutic treatment method. In this embodiment, genetic information encoding a transgene, either homologous or heterologous to the patient (such as the human opioid peptide enkephalin), is delivered to the epidermis and/or dermis by AAV and taken up by the primary sensory nerve endings. This genetic information is transported to the cell body in the DRG or TG to result in expression of the transgene that reduces pain by decreasing electrical excitability, and/or by modulating other components underlying pain, such as receptors, neurotransmitters, ion channels, second messenger systems, and biochemical mediators of inflammation. Other examples of transgenes include but are not limited to opioid peptides in a general sense such as dynorphin, orphanin, POMC (and its cleavage products gamma-MSH, alpha-MSH, CLIP, CTH, gamma-LPH, beta-LPH, beta-endorphin). Pertinent background references include Neuropsychopharmacology, Chapter 3, Opioid Peptides and their Receptors: Overview and Function in Pain Modulation, McNally and Akil, pp. 35-46 (ACNP, 2002), which is incorporated by reference herein in its entirety. Other possible proteins to be provided would be GABA (gamma-aminobutyric acid), endomorphin-1 and endomorphin-2.

Another possible protein therapy is the delivery and expression of magnetically sensitive ion channels, preferably by virus. Pertinent background references include Chen et al., Science 347(6229(:1477-80 (2015), which is incorporated by reference herein in its entirety. Similar in concept to optogenetics, but utilizing magnetism rather than light to trigger the alteration in ion transport, these specially engineered proteins can be delivered to cells and then magnets are used to either depolarize or hypopolarize the expressing cells, as desired. By providing these proteins to the nerves of the sensory unit using the methods of the present invention, a desired effect is achieved, such as the reduction of pain, either acute or chronic.

In another embodiment, this approach is taken to enable a gene silencing treatment method. In this embodiment, genetic information that encodes for knockdown of an endogenous protein (such as RNAi targeted against the Navl.7 voltage-gated sodium ion channel) or multiple endogenous proteins is delivered to the epidermis and/or dermis by AAV and taken up by the primary sensory nerve endings. This genetic information is transported to the cell body in the DRG or TG to result in reducing levels of an endogenous protein target that results in reducing pain by decreasing electrical excitability, and/or by modulating other components underlying pain, such as receptors, neurotransmitters, ion channels, second messenger systems, and biochemical mediators of inflammation. Other examples of knockdown methodology include but are not limited to approaches based on CRISPR, TALENs, microRNA, and zinc-finger nucleases.

Other examples of endogenous protein targets that could be knocked down in expression include but are not limited to the other voltage-gated sodium ion channels, Navl.3, Navl.8 and Navl.9 and the calcium voltage-gated ion channels Cav2.2 and Cav3.2. Other specific knockdown targets include P2X, DOR, NR2B, mACHR subtype M2, mAChR subtype M3, mACHR subtype M4, NTS2, Homer1, Shank1, TRPV1, DREAM, CCR2, GDNF, NR2B, PKCγ, Toll-like receptor 4, NR1 subunit of NMDA, and connexin 43. Utilizing knock down of one or more genes such as these, a desired effect is achieved within the sensory unit, such as the reduction of pain, either acute or chronic.

In another embodiment, such a method or configuration may be used to enable a receptor-modifying or chemogenetic approach to altering sensory unit function. In such an embodiment, genetic information encoding a ligand-activated G-protein coupled receptor (such as the hM4 Designer Receptors Exclusively Activated by Designer Drugs (“DREADD”) protein) is delivered to the epidermis and/or dermis by AAV and taken up by the primary sensory nerve endings. This genetic information is transported to the cell body in the DRG or TG to result in expression of the ligand-activated G-protein coupled receptor that can then be modulated by systemic administration (intravenous or oral) of its ligand (such as the ligand for hM4 DREADD protein, clozapine-N-oxide (“CNO”)). Modulation of the G-protein coupled receptor by its ligand can reduce pain by decreasing electrical excitability, and/or by modulating other components underlying pain, such as receptors, neurotransmitters, ion channels, second messenger systems, and biochemical mediators of inflammation.

In another embodiment, genetic information is delivered to the epidermis and/or dermis by AAV and taken up by the primary sensory nerve endings where the genetic information has a known impact on the itch reaction or targets neuronal responses that suppress itch in a generalized sense. This genetic information is transported to the cell body in the DRG or TG to result in reduction of itch. Examples of genetic information include but are not limited to i) light-activated ion transporter proteins (such as iC++) that are expressed at the cell body and trafficked back down to the primary sensory nerve endings in skin, where they may be modulated by intradermal light delivery to reduce itch, and ii) knockdown of itch-related genes (such as gastrin-releasing peptide (GRP) and natriuretic polypeptide b (Nppb)) that selectively reduce itch sensation without modulation of touch, pain and proprioceptive sensations.

In another embodiment, genetic information encoding either a transgene or knockdown of an endogenous protein to reduce pain or itch are delivered to the epidermis and/or dermis by any viral vector capable of being taken up by the primary sensory nerve endings. Examples include but are not limited to all wild-type and engineered variants of adeno-associated virus (AAV), herpes simplex virus (HSV), adenovirus, lentivirus, and rabies virus. In particular, it has been noted that some viral strains are more efficient at the skin-directed administration than others. For AAV, it has been noted that AAV that has coat proteins from type 6 (denoted AAV6), type 1 (denoted AAVA1), and type 8 (denoted AAV8) are more efficient than other types in resulting in transduction after the kind of administration described herein.

The present data also provides the ability to use either parent AAV strains or those that have been engineered to be self-complementary within the methods of the present invention. Briefly, self-complementary AAV strains (scAAV) are those that have been genetically engineered to hypothetically allow more efficient transduction, as upon infection the host cell does not need to produce the second strand of the DNA for the replication stage. Instead, the two halves of the scAAV will associate with each other, thus forming the double stranded DNA molecule needed to start replication and transcription. The downside to the use of such strains is the lower transport capacity, as scAAV can carry only about 2.4 kb of genetic information, which parent strains can deliver from about 4.7-6 kb of information. However, functional opsin proteins can be engineered that will meet these size requirements, as disclosed herein.

In another embodiment, genetic information encoding either a transgene or knockdown of an endogenous protein to reduce pain or itch are delivered to the epidermis and/or dermis by any nucleotide delivery approach capable of being taken up by the primary sensory nerve endings. Examples include but are not limited to small interfering RNA, naked DNA, and liposome encapsulated nucleotides.

In another embodiment, genetic information encoding either a transgene or knockdown of an endogenous protein to reduce pain or itch are delivered to the epidermis and/or dermis by a stem cell approach. An example includes but is not limited to the use of human embryonic stem cell-derived epithelial keratinocytes that express the blue light-sensitive, or light-responsive, chloride ion channel, iC++, that are activated by intradermal light delivery to inhibit action potential generation in primary sensory nerve endings.

In another embodiment, a purified recombinant protein or combination of multiple recombinant proteins are delivered directly to the epidermis and/or dermis to be taken up by primary sensory nerve endings or cutaneous cells to modulate pain or itch.

In another embodiment, genetic information encoding either a transgene or knockdown of an endogenous protein to reduce pain or itch are delivered directly to the cells of the epidermis and/or dermis. An example includes but is not limited to delivery of genetic information encoding the blue light-activated chloride ion channel, iC++, directly to keratinocytes such intradermal light delivery can inhibit action potential generation in primary sensory nerve endings.

Delivery of genetic information to the epidermis and/or dermis in the configurations described above can be achieved through many methods. Examples of cutaneous or at least partially through the skin delivery configurations include but are not limited to subcutaneous injections, transdermal injections, intradermal injections, topical application, and enhanced transfer of genetic information by electroporation, ultrasound treatment, microabrasion and coated gold microparticle delivery.

Such configurations described above may be applied to specific acute and chronic pain disorders including but not limited to neuropathic pain, trigeminal neuralgia, complex regional pain syndrome (CRPS, also known as reflex sympathetic dystrophy (RSD) or reflex neurovascular dystrophy (RND)), post-surgical pain, somatic pain, diabetic peripheral neuropathy, sciatica, and post-herpetic neuralgia. It should be noted that chronic pain is commonly defined as any pain lasting more than 12 weeks.

Such configurations described above may be applied to specific acute and chronic itch disorders including but not limited to post-herpetic itch, atopic eczema, and contact dermatitis.

In another embodiment, genetic information encoding for knockdown of both pain-related genes and HSV genes may be achieved, as there may be a concern with post-herpetic itch and post-herpetic neuralgia that viral gene therapy may result in reactivation of the HSV genes.

In another embodiment, adeno-associated virus variants may be engineered with enhanced uptake from nerve endings in the epidermis and/or dermis. Example of this process include but are not limited to directed evolution of AAV capsids, sexual PCR, and the Cre recombination-based AAV targeted evolution (CREATE) system. Here, multiple rounds of injection into epidermis and/or dermis followed by extraction at the DRG, would be performed to isolate/create a vector with enhanced primary sensory neuron uptake.

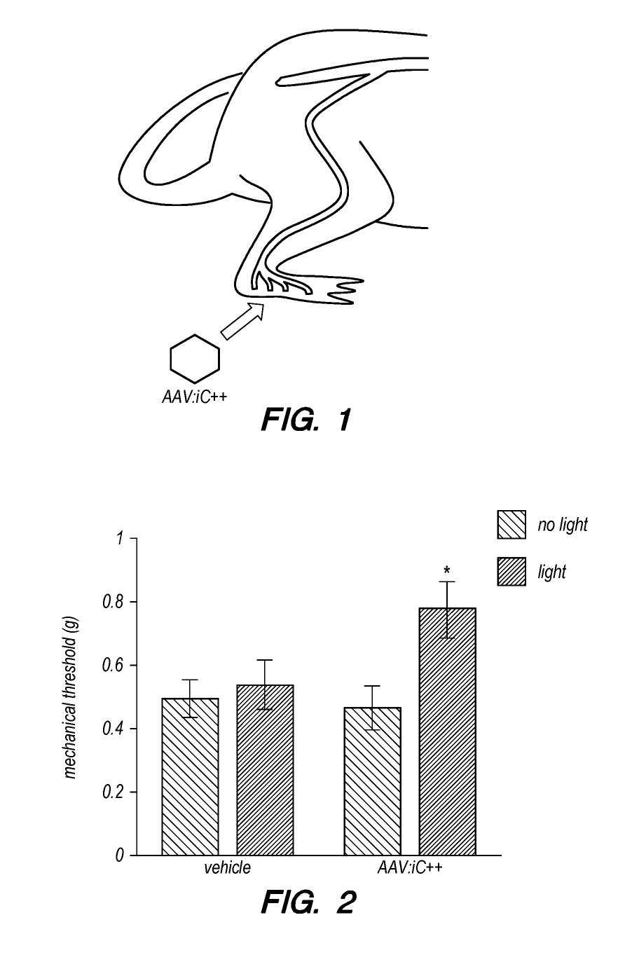

Referring to FIGS. 1 and 2 generally, in initial studies, injection subcutaneously of AAV expressing iC++ inhibits pain through optogenetics.

As noted above, pain management is a major concern in modern medicine. For example, post-surgical pain is the largest pain market ($5.9 billion in 2010) with >100 million surgeries per year requiring post-surgical pain treatment (majority are Percocet and Vicodin). There is a large unmet need with 40% of patients reporting inadequate pain relief, mainly due to the limitation of side effects of drugs that include dizziness, sedation, nausea, respiratory depression and euphoria. In addition, opioids are highly addictive with >2 million people addicted in the United States, leading to >20,000 lethal overdoses (2× more than heroin lethal overdoses). Furthermore, in many instances opioids are the gateway drugs for other illegal narcotics. Post-surgical pain is attractive for optogenetic therapy as much of the pain arises at the skin surface due to C and Adelta fiber sensitization and ectopic activity. Here a light emitting optogenetic bandage would be ideal to turn off this activity locally without off-target effects. Furthermore, the indication requires therapy of only weeks (US20160030765 not years) which would reduce preclinical and clinical development timelines. U.S. patent application publication to Towne et al describes optogenetic configurations suitable for practice of various methods described herein; this reference is incorporated by reference herein in its entirety.

In one embodiment, a system and method may be utilized to modulate pain through cutaneous viral gene transfer of DNA or mRNA as follows.

One objective is to deliver the genetic information of an optogenetic protein, such as iC++, through the skin to treat post-surgical pain using a virus. The optogenetic protein may be expressed in the skin and/or nerve endings through topical, subcutaneous or intradermal delivery of virus carrying the DNA or mRNA. Ideally, the formulation may be rubbed onto the skin (topical). After gene delivery, the iC++ protein may be activated in one embodiment by blue light to inhibit spontaneous and sensitized activity in C- and Adelta-fibers to reduce pain following surgical incisions. Light may be delivered intradermally through a light emitting optogenetic bandage. The viral DNA or mRNA may be delivered through formulations that comprise other excipients as needed for the chosen delivery method. Variations of such approach are described above, including but not limited to treatment of other pain disorders, use of other optogenetic proteins, use of other therapeutics genes and knockdown approaches, and treatments of itch using virally delivered genetic information.

In one embodiment, a system and method may be utilized to modulate pain through cutaneous non-viral gene transfer of DNA or mRNA as follows.

One objective is to deliver the genetic information of an optogenetic protein, such as iC++, through the skin to treat post-surgical pain. The optogenetic protein may be expressed in the skin and/or nerve endings through topical, subcutaneous or intradermal delivery of DNA or mRNA. Ideally, the formulation may be rubbed onto the skin (topical). After gene delivery, the iC++ protein may be activated in one embodiment by blue light to inhibit spontaneous and sensitized activity in C- and Adelta-fibers to reduce pain following surgical incisions. Light may be delivered intradermally through a light emitting optogenetic bandage. The DNA or mRNA may be delivered through formulations such as cationic polymers (e.g. polyethylenimine or gelatin), cationic lipids, protamine, apatite, or gold particles. Likewise, gene transfer of DNA or mRNA may be achieved by physical methods such as electroporation, ultrasound treatment or microabrasion. In the case of mRNA delivery, formulations may deliver mRNA directly to nerve endings, that contain mRNA translation machinery, and allow production of protein directly at nerve endings without requiring gene delivery to the nucleus at the TG or DRG cell body. Variations of such approach are described above, including but not limited to treatment of other pain disorders, use of other optogenetic proteins, use of other therapeutics genes and knockdown approaches, and treatments of itch.

Referring to FIGS. 1, 2, and 3, various aspects of configurations and methods are illustrated for transdermal delivery of AAV expressing an inhibitory opsin to increase mechanical threshold levels in uninjured mice.

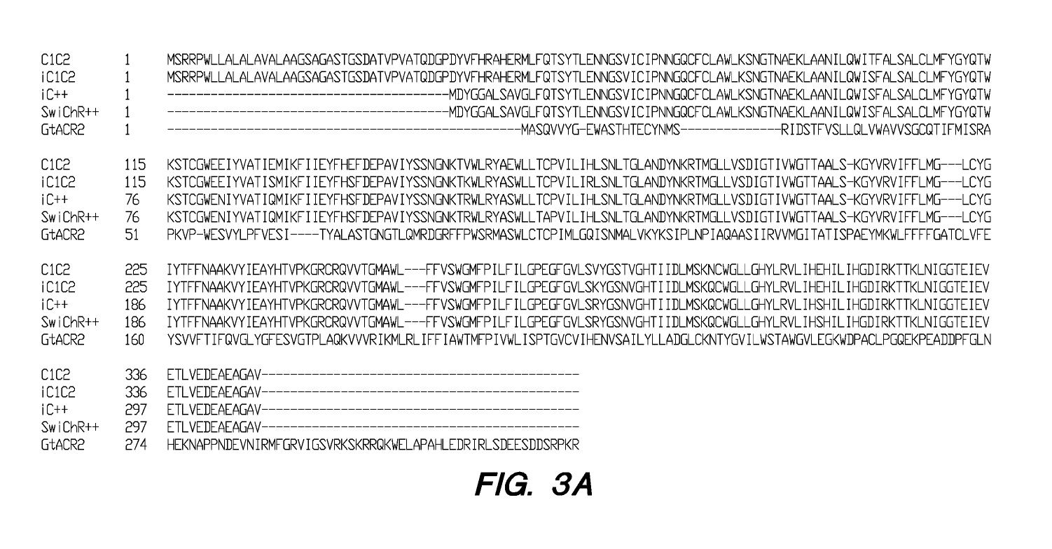

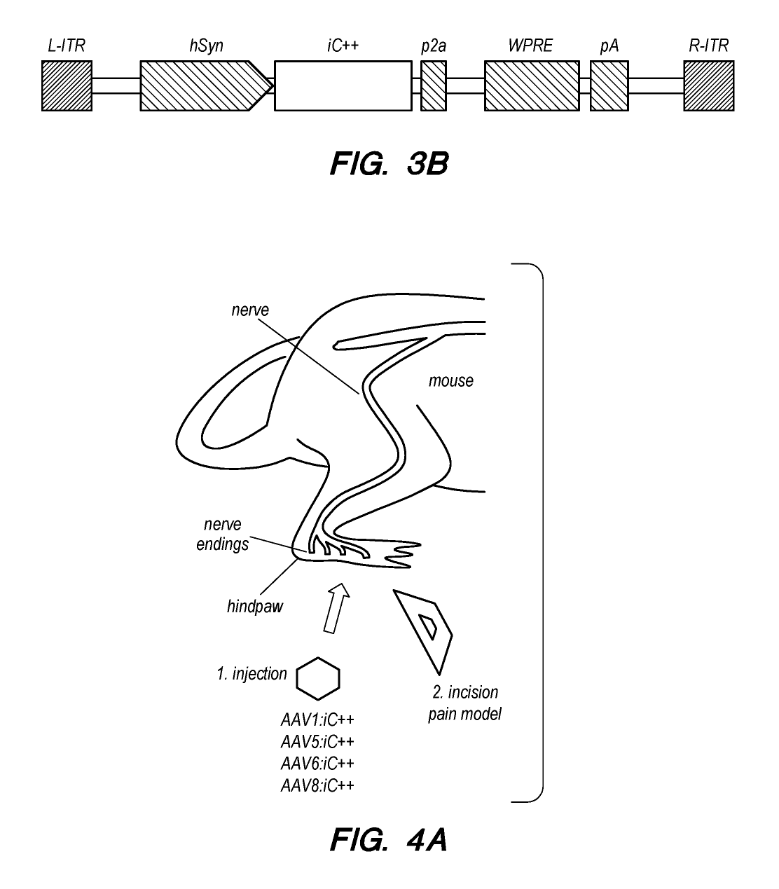

Referring to FIGS. 3A and 3B, in one embodiment an adeno-associated virus (AAV) expression plasmid was constructed that contains the iC++ transgene under control of the human synapsin promoter using standard cloning methods. iC++ is a synthetic blue-light sensitive chloride channel that has previously been used to inhibit neural activity in vivo. Reference is made to Berndt A, Lee S Y, Wietek J, Ramakrishnan C, Steinberg E E, Rashid A J, Kim H, Park S, Santoro A, Frankland P W, Iyer S M, Pak S, Ahrlund-Richter S, Delp S L, Malenka R C, Josselyn S A, Carlen M, Hegemann P, Deisseroth K. Structural foundations of optogenetics: Determinants of channelrhodopsin ion selectivity. Proc Natl Acad Sci U S A. 2016 Jan 26;113(4):822-9, which is incorporated by reference herein in its entirety. The cassette also contains the 2A peptide (p2a) sequence, the woodchuck hepatitis virus posttranscriptional regulatory element (WPRE), a poly adenylation (polyA) signal and two inverted terminal repeats (ITRs). This expression cassette can be packaged into multiple AAV serotypes depending upon the packaging plasmid used. In this example the expression cassette was packaged in AAV serotype 8.

AAV serotype 8 expressing iC++ was generated through an adenovirus-free triple transfection method as described previously; reference is made to Xiao X, Li J, Samulski R J. Production of high-titer recombinant adeno-associated virus vectors in the absence of helper adenovirus. J Virol. 1998 March;72(3):2224-32; and Matsushita T, Elliger S, Elliger C, Podsakoff G, Villarreal L, Kurtzman G J, Iwaki Y, Colosi P. Adeno-associated virus vectors can be efficiently produced without helper virus. Gene Ther. 1998 July;5(7):938-45; each of which is incorporated by reference in its entirety herein. Briefly, HEK 293 cells were transfected via calcium phosphate with the iC++ expression plasmid described above, the packaging plasmid that contains the Rep (replication) and Cap (capsid) genes required to recognize the ITRs and package their flanked sequences into the virion, and the helper plasmid that supplies the remaining adenovirus proteins required for AAV construction. Forty-eight hours after transfection, the packaged AAV particles were liberated from the nucleus through cell lysis and the homogenates added to a cesium chloride gradient where ultracentrifugation separated the AAV particles from cell debris. Quantitative polymerase chain reaction (PCR) using primer pairs against sequences in the expression cassette were used to titrate vector particles (viral genomes per milliliter, vg).

Referring back to FIG. 1, 1×10¹¹ vg of AAV serotype 8 expressing iC++ suspended in 5 uL of phosphate buffered saline (PBS) was injected into the glabrous plantar skin located on the hind paw of anesthetized, 6 week old, C57B17 mice using a 1 mL BD Ultrafine insulin syringe with a 6 mm by 31G needle (n=10 per group). The needle was inserted with the bevel facing upwards at the most caudal part of the footpad, and slowly passed forwards while remaining parallel to, and just beneath, the surface of the skin. Once the needle tip reached the midpoint of the foot the injection was performed and the needle was removed slowly to prevent leakage. Control animals were injected using the same method but with the vehicle only. Animals were returned to their home cage for future experimentation.

The mechanical threshold levels of the mice were investigated 3 weeks after by the up-down method of von Frey testing; reference is made to Chaplan S R, Bach F W, Pogrel J W, Chung J M, Yaksh T L. Quantitative assessment of tactile allodynia in the rat paw. J Neurosci Methods. 1994 July;53(1):55-63, which is incorporated by reference in its entirety herein. Mice were allowed habituate to the test setup for 30 minutes and then von Frey filaments of various forces were applied to the bottom of the injected paw to ascertain withdrawal thresholds. Poking was performed in the presence or absence of 2 mW/mm² blue light delivered through an optical fiber held 1 cm from the skin surface supplied from a laser source (471 nm). Referring to FIG. 2, we observed that AAV treated animals had increased mechanical threshold levels in response to blue light, unlike vehicle treated animals that remained unchanged. Therefore, intradermal delivery of an AAV expressing iC++ facilitates light-mediated modulation of mechanical threshold levels in mice.

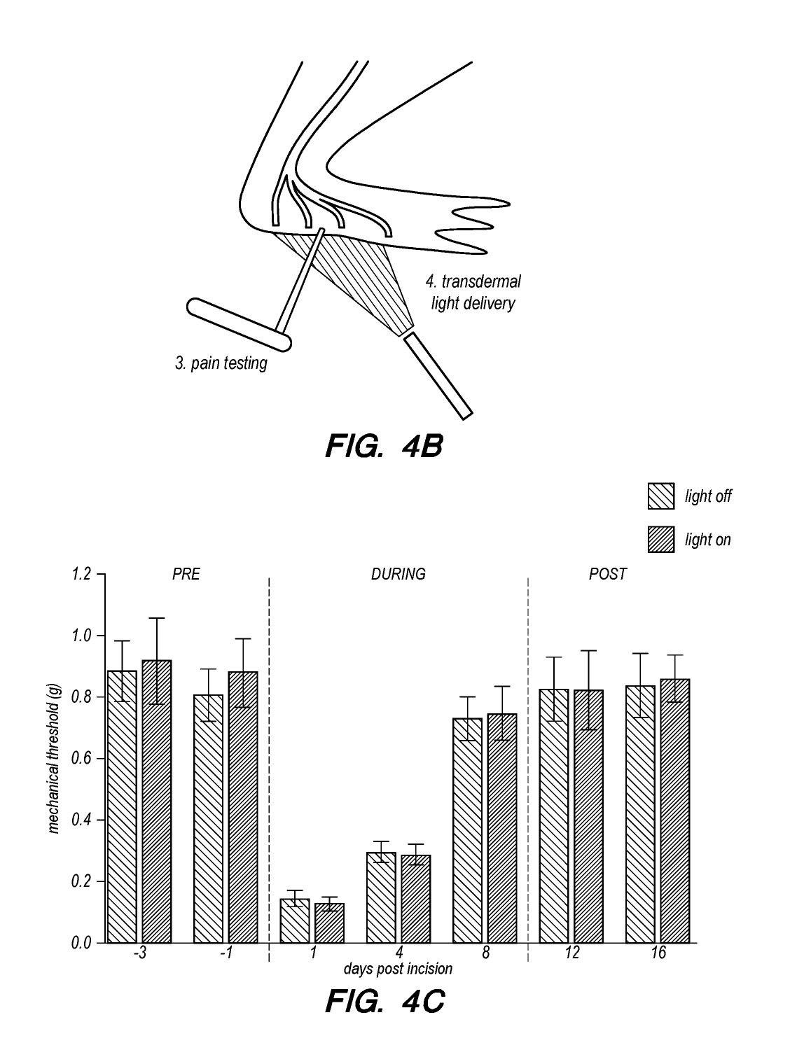

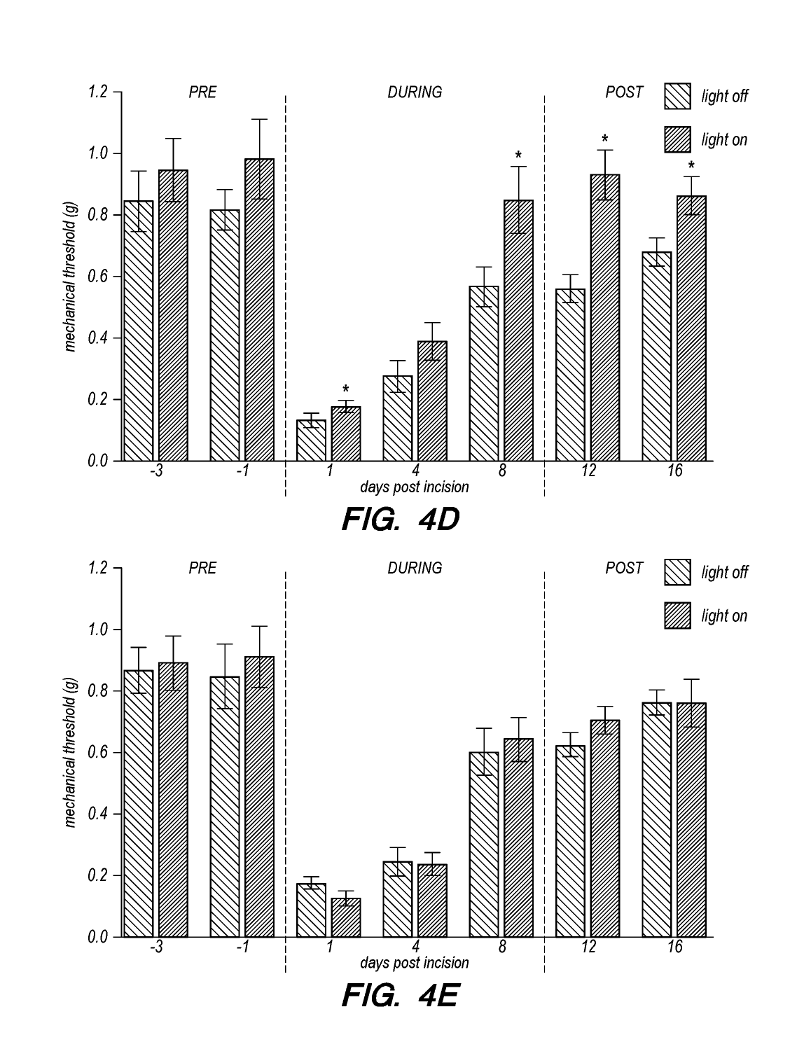

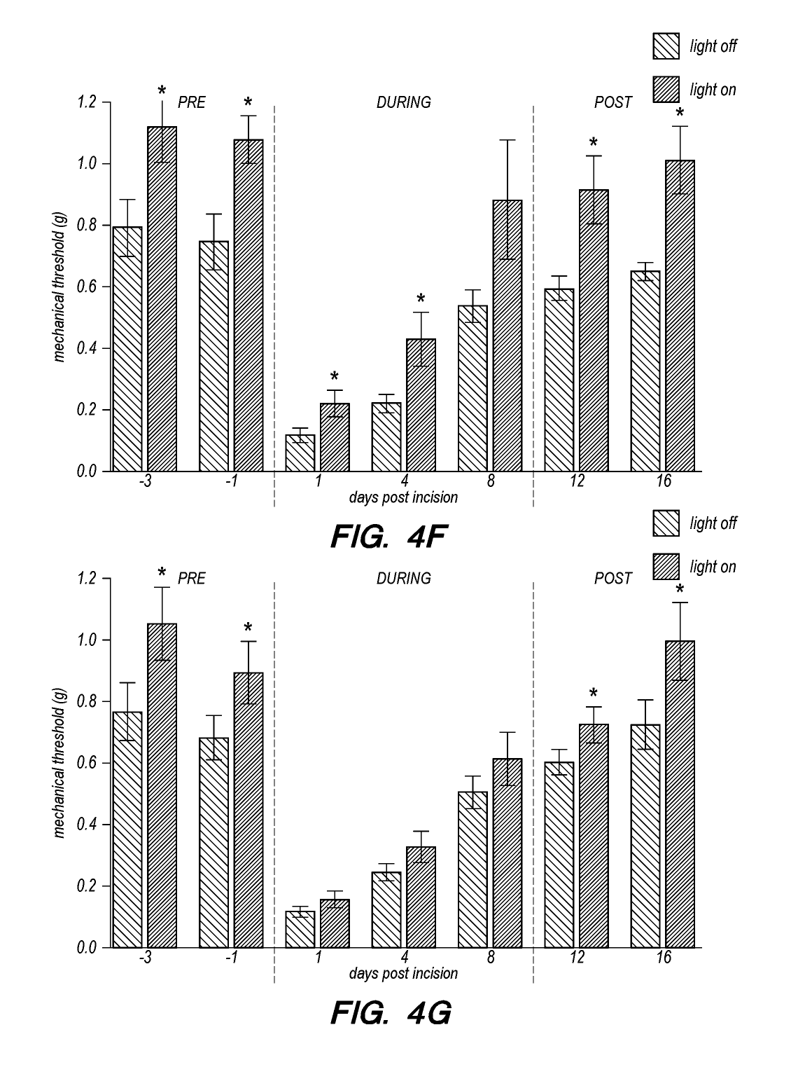

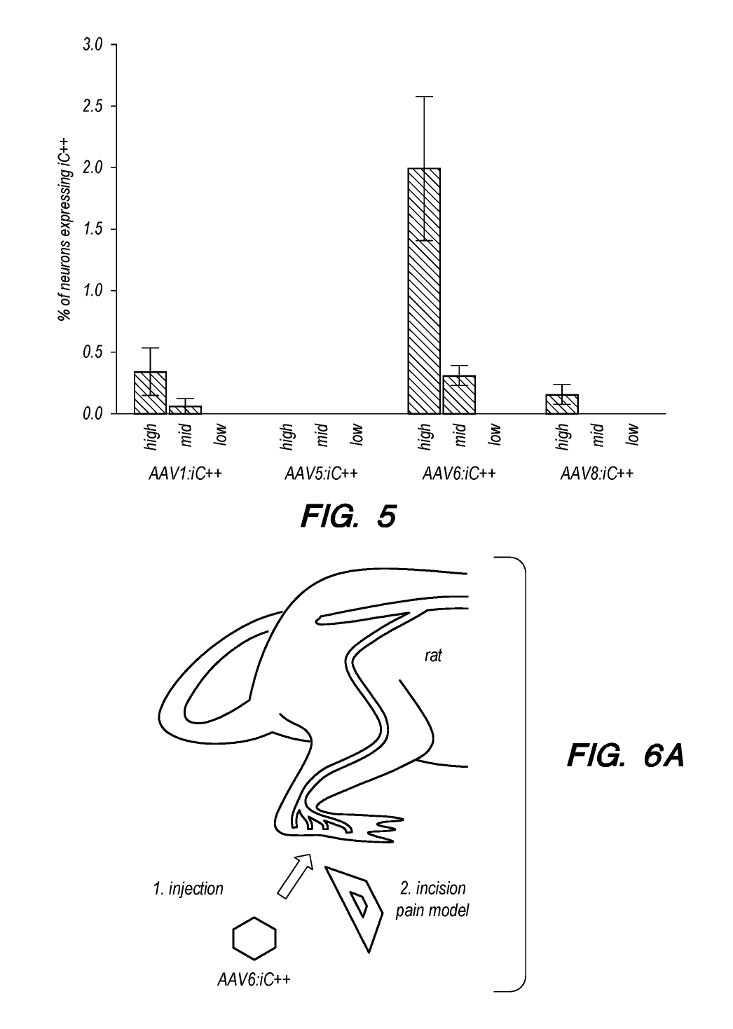

Referring to FIGS. 4A-5, various aspects of configurations and methods are illustrated for transdermal delivery of AAV expressing an inhibitory opsin which reduces post-surgical pain in mice and is serotype dependent. In this embodiment, the therapeutic effect of light application after intradermal injection of various AAV serotypes expressing iC++ was tested in a mouse model of post-surgical pain. Four different serotypes of AAV vectors expressing iC++ were generated as described above in reference to FIGS. 1-3B using packaging plasmids for AAV serotype 1, 5, 6 and 8. Referring to FIG. 4A, 6 week old C57B16 mice were injected with 1.4×10¹¹, 8.8×10¹⁰, 3.8×10¹⁰ and 1.9×10¹¹ vg of AAV serotype 1, 5, 6 and 9 expressing iC++, respectively, using the surgical method described in example 1 (n=10 per group). A vehicle injected group was generated as a control.

Referring to FIG. 4B, three weeks later, mechanical threshold levels of the mice were determined in the presence or absence of blue light, as described above in reference to FIGS. 1-3. Referring to FIGS. 4C-4G, we observed light-mediated increases in mechanical threshold levels with AAV serotype 6 and 8, but not in serotypes 1, 5 and the vehicle. This assay was repeated 2 days later with the same result.

Mice were incised with a scalpel blade on the plantar surface of the injected hind paw to generate the model of post-incisional pain as described previously; reference is made to Pogatzki E M, Raja S N. A mouse model of incisional pain. Anesthesiology. 2003 October;99(4):1023-7, which is incorporated by reference herein in its entirety. Briefly, mice were anesthetized with 1.5% to 2.5% isoflurane delivered via a nose cone. After antiseptic preparation of the hind paw with 10% povidone-iodine solution, a 5 mm longitudinal incision was made with a number 11 blade through the skin and fascia of the plantar foot. The incision was started 2 mm from the proximal edge of the heel and extended toward the toes. The underlying muscle was elevated with a curved forceps, leaving the muscle origin and insertion intact. The skin was apposed with a single suture of 8-0 nylon. The mouse was then returned to cage for recovery.

One, 4, 8, 12 and 14 days later the mice were assayed for mechanical threshold levels in the presence or absence of light. We observed that light application to the paw of AAV serotype 1, 6 and 8 mice resulted in decreasing mechanical allodynia caused by the incision, whereas AAV serotype 5 or vehicle injected animals had no change. Therefore, intradermal delivery of certain AAV serotypes expressing iC++ facilitates light-mediated inhibition of post-surgical pain in mice.

A cohort of 6 week old naive C57B16 mice was injected with the AAV vectors described above for purposes of histological analysis (n=3 per group). Here, the same serotypes and doses were injected, however, two additional doses for each serotype were examined at ⅕ and 1/25 dilutions of the original dose. These mice were sacrificed 4 weeks later and the dorsal root ganglia (DRG) from vertebral levels L4 and L5 were analyzed for transduction. Here, mice were transcardially perfused with 4% paraformaldehyde in PBS and dissected tissue post-fixed overnight in the same solution at 4 degrees Celsius. The DRG were cryoembedded and cut on a cryostat at 12 um thickness. Sections were stained in parallel with an antibody against iC++ detected by a secondary antibody in the green channel (488 nm) and a fluorescent Alexa Fluor 594 conguated to the Nissl stain in the red channel (594 nm). Nissl is a neuronal specific stain and was used to quantify the percentage of total DRG neurons expressing iC++ (FIG. 5). We observed that AAV serotype 1, 6 and 8 transduced neurons of the DRG whereas AAV serotype 5 did not. Furthermore, AAV serotype 6 was the most efficient at retrograde transport, with a relatively low dose of the serotype transducing more cells than the other serotypes.

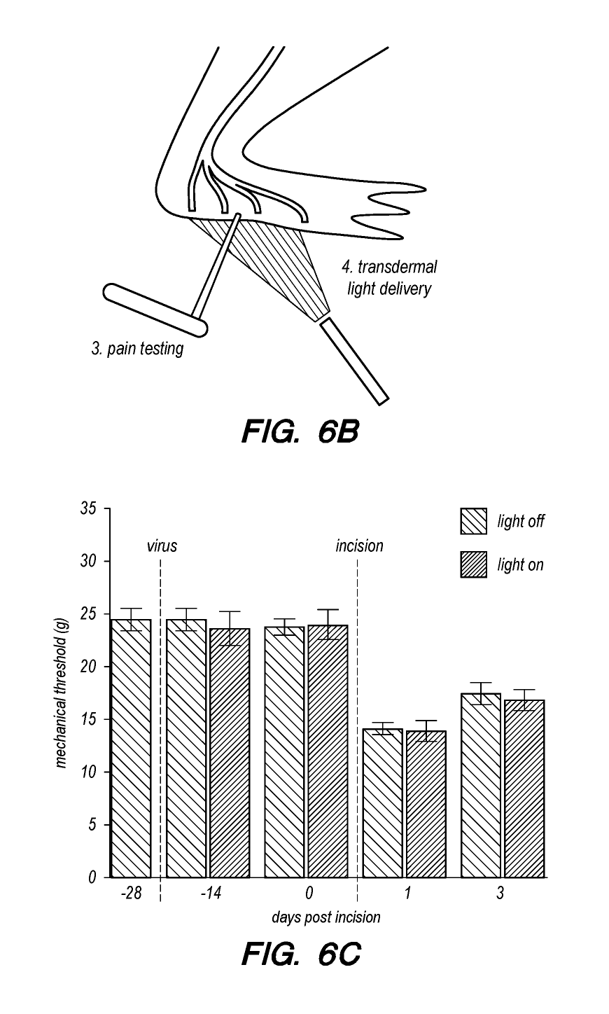

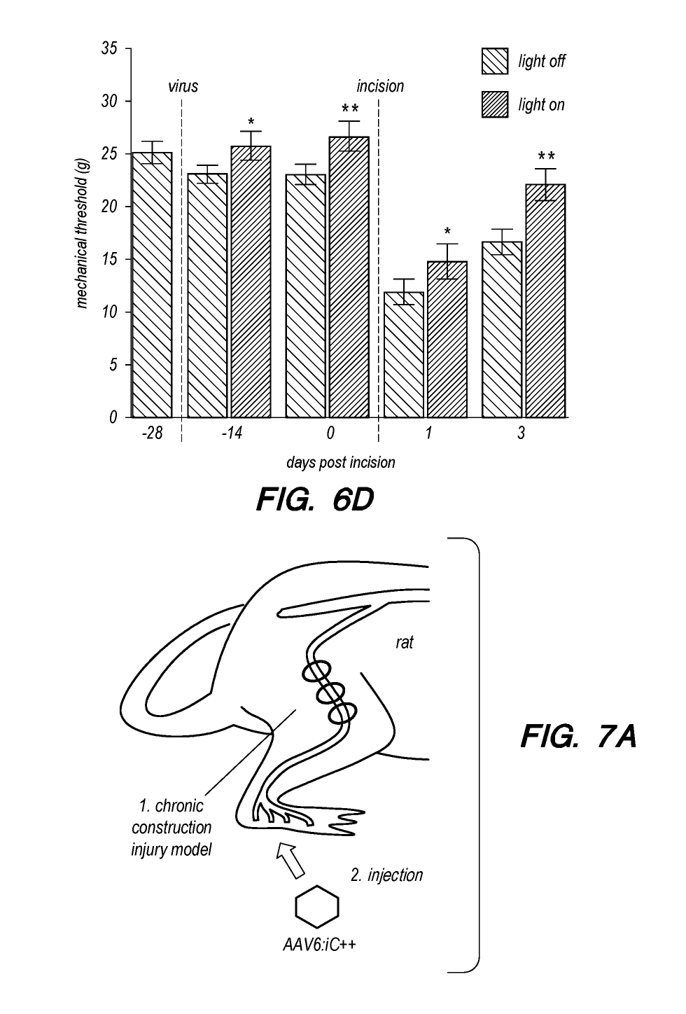

Referring to FIGS. 6A-6D, various aspects of configurations and methods are illustrated for transdermal delivery of AAV expressing an inhibitory opsin to reduce post-surgical pain in rats. In this embodiment, the therapeutic effect of light application after intradermal injection of AAV serotype 6 expressing iC++ was tested in a rat model of post-surgical pain. The experiment was performed similar to that described in reference to FIGS. 4A-5, except that only AAV serotype 6 was examined, and that the experiment was performed in rats. Referring to FIGS. 6A and 6B, 2×10¹¹ vg of AAV serotype 6 suspended in 20 uL of PBS was delivered into the hind paw of 10 week old Sprague Dawley rats as described in reference to FIGS. 1-3B. A vehicle injected group was also generated as a control. Mechanical threshold levels were determined in the presence or absence of blue light at 2 and 4 weeks post-virus injection. Referring to FIGS. 6C and 6D, we observed that light application increased mechanical threshold levels for the vector injected group but not the vehicle injected group. Rats were then incised on the hind paw to generate a model of post-incisional pain using the method described in reference to FIGS. 4A-5. Mechanical threshold levels were determined in the presence or absence of blue light at 1 and 3 days post-incision. We observed that light application increased mechanical threshold levels for the vector injected group but not the vehicle injected group. Therefore, light application following intradermal delivery of AAV6 expressing iC++ inhibits post-surgical pain in rats.

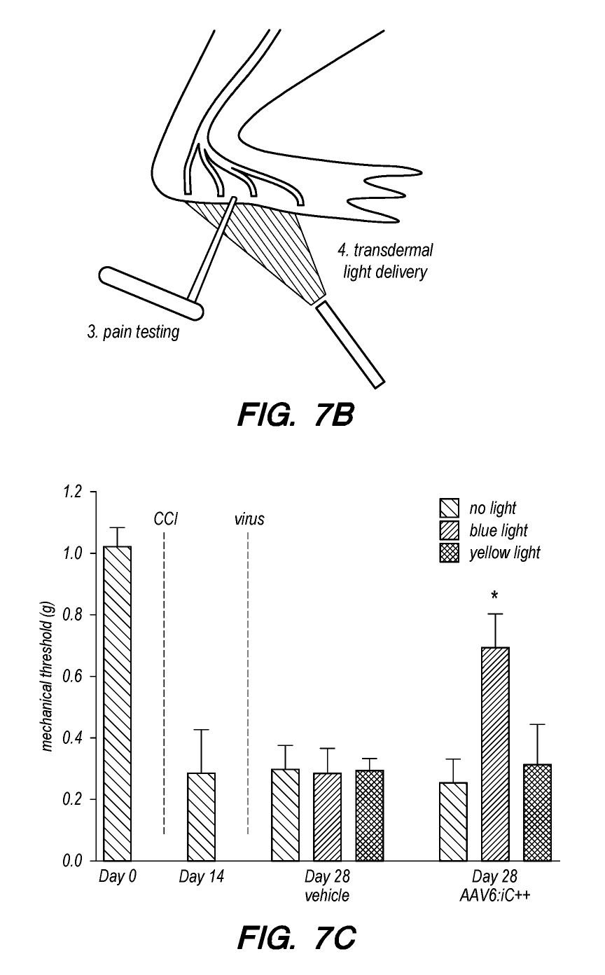

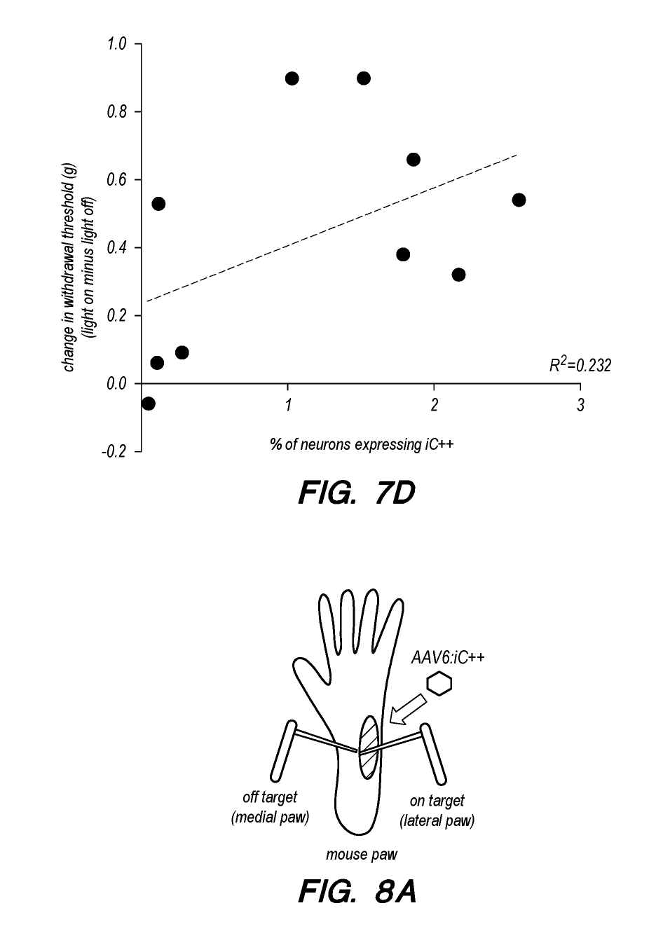

Referring to FIGS. 7A-7D, various aspects of configurations and methods are illustrated for transdermal delivery of AAV expressing an inhibitory opsin to reduce neuropathic pain in mice. In this embodiment, the therapeutic effect of light application after intradermal injection of AAV serotype 6 expressing iC++ was tested in a mouse model of neuropathic pain. 6 week old C57B16 mice were assayed for mechanical threshold levels as described above in reference to FIGS. 1-3B. Referring to FIGS. 7A-7C, next, neuropathic pain phenotype was generated via chronic constriction injury as described previously; reference is made to Bennett G J, Xie Y K. Pain. 1988 April;33(1):87-107. A peripheral mononeuropathy in rat that produces disorders of pain sensation like those seen in man, which is incorporated by reference herein in its entirety. Briefly, mice were anesthetized with 1.5% to 2.5% isoflurane delivered via a nose cone. After antiseptic preparation of the skin on the thigh, a 1 cm incision was made to expose the quadriceps muscle. The sciatic nerve was exposed through blunt dissection and then constricted through placement of three 6-0 chromic gut sutures tied loosely at 1 mm distances from each other. The skin was sutured closed and the animal allowed to recover. Mice were assessed for mechanical threshold levels, revealing a dramatic decrease in mechanical threshold levels indicative of neuropathic pain. 1×10¹¹ vg of AAV serotype 6 expressing iC++ suspended in 5 uL of PBS was intradermally injected into the paw as described in example 1. A vehicle injected group was generated as a control. Mechanical threshold levels were assessed three weeks later in the presence or absence of blue light as described in example 1. In addition, animals were also tested in the presence of 2 mW/mm² yellow light, supplied through an optical fiber connected to a laser emitting 594 nm light. Yellow light does not activate the iC++ channel and serves as a control for the wavelength specificity. We observed that blue light application increased mechanical threshold levels for the vector injected group but not the vehicle injected group. Furthermore, yellow light had no effect on mechanical threshold levels. Therefore, light application following intradermal delivery of AAV6 expressing iC++ inhibits neuropathic pain in mice.

After the behavioral experiment, the AAV6 injected mice were sacrificed and transduction rates assessed as described above in reference to FIGS. 4A-5. Referring to FIG. 7D, these transduction rates were plotted against the difference in mechanical threshold for the AAV6 injected mice between blue light and no light i.e. the therapeutic effect. We observed there was a trend (R²=0.223) for a correlation between transduction rate and therapeutic effect.

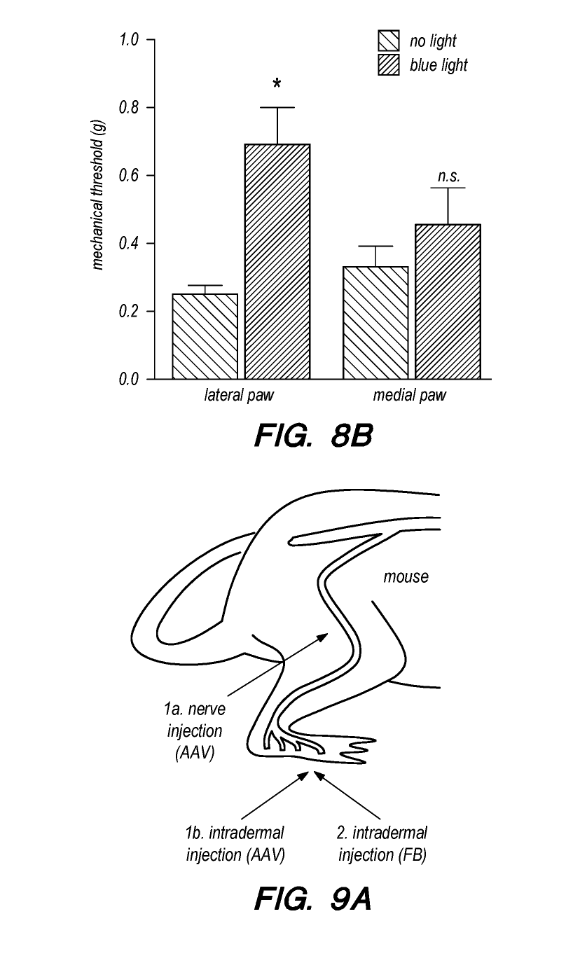

Referring to FIGS. 8A-8B, various aspects of configurations and methods are illustrated for transdermal delivery of AAV resulting in restricted expression and restricted therapeutic effect in the transgene. In this embodiment, a cohort of mice was generated to examine the restricted therapeutic effect of intradermal delivery via histological analysis. Intradermal injections of AAV serotype 6 expressing iC++ were made in mice with neuropathic pain as described above in reference to FIGS. 6A-6D. Referring to FIG. 8A, note that for vector administration, virus was injected into the lateral plantar surface of the hindpaw, and no viral solution was observed to leak to the medial plantar surface of the hindpaw. Two weeks following virus injection, animals were assessed for mechanical threshold levels in the presence or absence of blue light on both the lateral and medial surface of the hindpaw. Referring to FIG. 8B, as expected, we observed that blue light reduced pain when the animals were tested at the injection site (lateral paw). However, we did not observe an effect when the animals were tested on the medial paw.

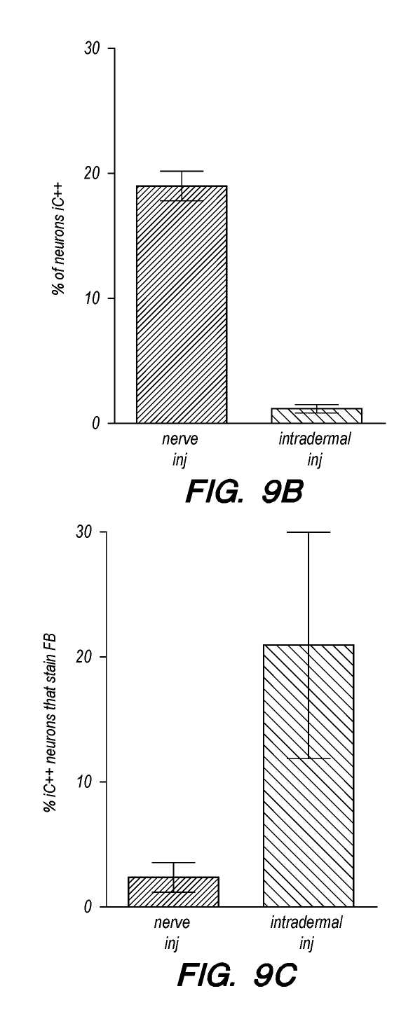

Referring to FIGS. 9A-9C, a cohort of mice was generated to examine the restricted expression of intradermal delivery via histological analysis. One cohort of mice (n=5) was injected with 1×10¹¹ vg of AAV6 expressing iC++ in 5 uL of PBS into the lateral plantar hindpaw as described in example 1. A second cohort of mice (n=5) was injected with 1×10¹¹ vg of AAV6 expressing iC++ in 5 uL of PBS into the sciatic nerve as described previously; reference is made to Iyer S M, Montgomery K L, Towne C, Lee S Y, Ramakrishnan C, Deisseroth K, Delp S L. Virally mediated optogenetic excitation and inhibition of pain in freely moving nontransgenic mice. Nat Biotechnol. 2014 March;32(3):274-8, which is incorporated by reference herein in its entirety. Briefly, the sciatic nerve was exposed as described in reference to FIGS. 7A-7D, injected with the vector solution using a 35G beveled needle, and then the skin closed with suture. Three weeks following injection, both cohorts were injected intradermally into the lateral plantar hindpaw with the fluorescent retrograde tracer, Fast Blue. Mice were sacrificed five days after and DRGs processed for histology as described in example 2. As expected, nerve injections resulted in higher levels of expression than intradermal injections with approximately 20% and 1.5% transduction, respectively. However, intradermal delivery resulted in higher levels of co-staining with the Fast Blue than nerve injections with approximately 21% and 2.5%, respectively (FIG. 8c). Taken together, this data demonstrates that transdermal delivery of AAV results in restricted expression and, subsequently, restricted therapeutic effect of a transgene.

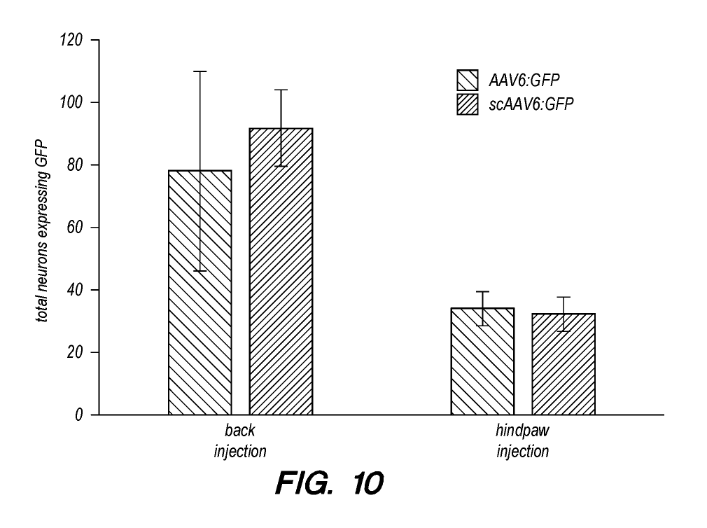

Referring to FIG. 10, various aspects of configurations and methods are illustrated for transdermal delivery of self-complimentary AAV resulting in equivalent levels of sensory neuron transduction. In this embodiment, self-complementary and single stranded AAV serotype 6 expressing green fluorescent protein (GFP) under control of the cytomegalovirus (CMV) promoter were purchased from ViroVek Inc (Hayward, Calif.). 4×10¹² vg of each vector was injected intradermally to the hindpaw of rats as described above in reference to FIGS. 4A-5 (n=4 per group). In addition, rats were injected with the same dose of vector intradermally onto the back of the animal, corresponding to the area above the scapula. Two weeks later animals were sacrificed and processed for histology as described in example 2. Here we observed expression of GFP in the DRG of the animals with both self-complementary and single stranded AAV serotype 6. These results demonstrate that both self-complementary and single stranded are capable of transducing DRG following intradermal delivery.

One embodiment is directed to a method of treating or preventing an undesired or lack of sensory response of a region of tissue by altering the function of the sensory unit that innervates that tissue region in a mammal comprising identifying the tissue region that has, will have, or is lacking the sensory response; intradermally or subcutaneously administering into the identified tissue region an adeno-associated virus having a coat protein selected from the group consisting of AAV strain 1, AAV strain 6, and AAV strain 8 where the viral genome encodes at least one exogenous protein; expressing the exogenous protein in the targeted sensory unit; and altering the function of the targeted sensory unit to treat or restore the sensory response because of the exogenous protein expression while not impacting the function of nearby sensory units. The exogenous protein may be a light-responsive protein and the method further may comprise exposing the targeted sensory unit to light. The light-responsive protein may be a stimulatory opsin. The stimulatory opsin may be selected from the group consisting of ChR2, C1V1-T, C1V1-TT, CatCh, VChR1-SFO, and ChR2-SFO. The light-responsive protein may be an inhibitory opsin. The inhibitory opsin may be selected from the group consisting of NpHR, eNpHR 1.0, eNpHR 2.0, eNpHR 3.0, Mac, Mac 3.0, Arch, ArchT, iChR, iC1C2, iC++, SwiChR++, and JAWS. The exogenous protein may be one which reduces pain by decreasing electrical excitability, or by modulating receptors, neurotransmitters, ion channels, second messenger systems, and biochemical mediators of inflammation that underlie pain. The exogenous protein may be selected from the group consisting of P2X, DOR, Nav 1.7, Nav 1.8, Cav 1.2, NR2B, mACHR subtype M2, mAChR subtype M3, mAChR subtype M4, NTS2, Homer1, Shank1, TRPV1, DREAM, CCR2, GDNF, NR2B, PKCγ, Toll-like receptor 4, NR1 subunit of NMDA, connexin 43, GABA, endomorphin, and a ligand associated G-protein. The undesired sensory response may be selected from the group consisting of acute pain, chronic pain, allodynia, ectopic pain, neuropathic pain, itch, and parathesia. The lack of sensory response may be anesthesia. The lack of sensory response may be a feeling of satiation. The AAV may be self-complementary.

Another embodiment is directed to a method of treating or preventing an undesired or lack of sensory response of a region of tissue by altering the function of the sensory unit that innervates that tissue region in a mammal, comprising identifying the tissue region that has, will have, or is lacking the sensory response; intradermally or subcutaneously administering into the identified tissue region an adeno-associated virus virus comprising a coat protein selected from the group consisting of AAV strain 1, AAV strain 6, and AAV strain 8 where the viral genome encodes at least one molecule that results in RNAi; expressing the RNAi molecule in the targeted sensory unit; and altering the function of the targeted sensory unit to treat or restore the sensory response because of the RNAi expression while not impacting the function of nearby sensory units. The undesired sensory response may be pain and the RNAi may be specific to reducing the expression of a protein selected from the group consisting of P2X, DOR, Nav 1.7, Nav 1.8, Cav 1.2, NR2B, mACHR subtype M2, mAChR subtype M3, mAChR subtype M4, NTS2, Homerl, Shankl, TRPV1, DREAM, CCR2, GDNF, NR2B, PKCγ, Toll-like receptor 4, NR1 subunit of NMDA, and connexin 43. The undesired sensory response may be selected from the group consisting of acute pain, chronic pain, allodynia, ectopic pain, neuropathic pain, itch, and parathesia. The lack of sensory response may be anesthesia. The lack of sensory response may be a feeling of satiation. The undesired sensory response may be chronic pain and the RNAi may be achieved through a ddRNAi specific to Nav 1.7. The AAV may be self-complementary.

Another embodiment is directed to a method of treating neuropathic pain in a region of tissue by altering the function of the sensory unit that innervates that tissue region in a mammal, comprising identifying the tissue region that has the undesired neuropathic pain; intradermally or subcutaneously administering into the identified tissue region an adeno-associated virus comprising a strain 6 coat protein where the genome encodes the opsin iC++; expressing iC++ in the targeted sensory unit and exposing the sensory unit to light; and reducing the neuropathic pain in the tissue region innervated by the sensory unit while not impacting the function of nearby sensory units. The AAV may be self-complementary.

Another embodiment is directed to a method of treating superficial somatic pain in a region of tissue by altering the function of the sensory unit that innervates that tissue region in a mammal, comprising identifying the tissue region that has the undesired superficial somatic pain; intradermally or subcutaneously administering into the identified tissue region an adeno-associated virus comprising a strain 6 coat protein where the genome that encodes iC++; expressing iC++ in the targeted sensory unit and exposing the sensory unit to light; and reducing the superficial somatic pain in the tissue region innervated by the sensory unit while not impacting the function of nearby sensory units. The AAV may be self-complementary.

With regard to transfer of genetic material into and/or across various layers of the skin, in certain embodiments it is useful to address the construct of the stratum corneum (“SC”). The skin is a highly immune-reactive tissue containing an abundance of antigen-presenting cells, especially within the epidermis. The stratum corneum further provides a physical barrier, preventing uptake of most topically applied entities. In order to enhance the penetration through the skin, the therapeutic agents disclosed herein may need to be either injected into the tissue directly or the stratum corneum may be removed or altered to provide more direct access to the underlying tissue. We will use the term delivery target to refer to the cell, cell component, or tissue that is to be infused with the therapeutic agent.

The stratum corneum may be removed employing methods that include but are not limited to tape stripping, dermabrasion, microdermabrasion, depilatory compounds (such as Nair), and laser ablation.

The permeability of the stratum corneum may be altered using methods which include but are not limited to: sonophoresis, iontophoresis, electroporation, microdermabrasion, microneedles, laser ablation, including fractionated laser ablation, and optoporation (laser induction).

Configurations for directly penetrating the epidermis and delivering an agent to the dermis include but are not limited to: needles, microneedles, and needle-free injection guns (ballistic delivery).

Tape stripping has been introduced as a means to remove and study SC cells whereby successive layers of the SC are removed by repeated application of adhesive cellophane tape to the skin surface. To practice the present invention, tape stripping may be performed by applying adhesive tape, such as 3M Blenderm surgical tape, to a patient's clean skin, then affixing it with firm pressure, and allowing it to settle for ≥10s before removing. This may be repeated approximately 5 times per intended location to remove at least a portion of the SC, often a substantial portion. Although there is non-negligible interindividual variability of this approach, intraoperative microscopy, such as confocal scanning laser microscopy (CLSM) using a VivoScope® product available from Lucid Instruments, Inc., may be employed to determine the endpoint. Under CLSM the SC may appear brighter than the underlying tissue due to their differing refractive indices and thus provide a robust means of detection of sufficient SC stripping.

Dermabrasion is a technique that typically employs a wire brush or a diamond wheel with rough edges (or burr or fraise) to remove the upper layers of the skin. The brush or burr rotates rapidly, taking off and leveling (or abrading or dermaplaning) the top layers of the skin. Microdermabrasion is a nonsurgical technique that is intended to affect only the SC, and often employs a moving liquid slurry or powder of corundum or aluminum oxide crystals that is pulled across the skin surface and into a vacuum chamber instead of a brush or wheel. The vacuum pressure and probe area may be adjusted to tailor the affect. For deep dermabrasion, the areas to be treated are cleaned and marked. A local anesthetic (such as lidocaine) is usually used to numb the skin before treatment, and ice packs are applied to the skin for up to 30 minutes. A freezing (cryogenic) spray may sometimes be used to harden the skin for deeper abrasions if the anesthetic and ice packs do not make the skin firm enough. Microdermabrasion technique is similar, but the procedure is much less severe, and may not require the use of anesthetic. However, the use of ice packs or cryogen to harden the skin may improve the efficacy and overall efficiency of the microdermabrasion process.

Depilatory compounds may also be used to remove at least a portion of the SC and hair on a patient's skin. Common active ingredients are calcium thioglycolate or potassium thioglycolate, which breaks down the disulfide bonds in keratin and weakens the hair so that it is easily scraped off where it emerges from the hair follicle. As the epidermis is also rich in keratin, contact with the depilatory chemical will cause irritation that in turn may serve to loosen the SC and provide for increased permeability for the therapeutic agent to penetrate into the delivery target or an intermediate tissue.

Laser ablation utilizes a laser to remove the upper layers of skin and either expose the delivery target or provide more direct access to it. Fractional ablation refers to a pattern of small ablation craters instead of entire surface stripping. The depth of laser ablation is a function of the laser wavelength (1) pulse energy, and pulse duration used. Holes as small as 100 μm in diameter and as deep as 1.5 mm may be created using, for example, an Er:YAG laser operating at a wavelength equal to about 2.94 μm with a pulse duration of 1 ms and the appropriate beam size on the tissue to achieve selective tissue removal above the ablation threshold of about 1 J/cm². An excessive pulse duration may lead to collateral heating and coagulation of the tissue surrounding the ablation crater, which may inhibit penetration into the delivery target. By way of nonlimiting example, a 40 μJ, 400 μs pulse of wavelength of about 1920 nm collimated light (absorption coefficient of water=about 126 cm⁻¹) from a Thulium fiber laser may be focused to a ø200 μm spot on the skin surface using a +200 mm focal length singlet lens with a 200 mm working distance and create a ø180×100 μm deep ablation crater. The laser spot may be moved over the skin surface manually or optically using a scanner, such as has been described in U.S. Pat. No. 7,824,896, which is incorporated by reference herein in its entirety.

Ballistic, “biolistic”, or gene gun delivery uses an adjustable pressure helium pulse to sweep DNA-, RNA-, or biomaterial-coated gold microcarriers from the inner wall of a small plastic cartridge directly into target cells. Socalled gene guns have been used to deliver plasmids to rat DRG neurons as a pharmacological precursor in studying the effects of neurodegenerative diseases such as Alzheimer's disease. An example of a commercially available system is the Helios® gene gun product, manufactured by Becton-Dickinson, Inc. It is a handheld device that provides rapid and direct transfection into a range of targets in vivo. Preparation of the biolistic particles may be performed using the general method described by Woods and Zito in doi:10.3791/675, which is incorporated by reference herein in its entirety.

The basis behind electroporation (or electrophoresis) is a cell's plasma membrane, or a collection of adjacent cells will stretch and become permeabilized when pulsed with an electric field so an agent may more readily enter a delivery target. Electroporation in vivo may be accomplished by placing at least a therapeutic agent on the skin, followed by pulsing of electrodes also placed on the skin within or adjacent to the area of skin containing the at least a therapeutic agent. A therapeutic agent may then be introduced into cells or other delivery target or an intervening tissue by possibly creating transient permeability or even the creation of pores.

Ionophoration (or iontophoresis) is similar to electroporation; however, it uses weaker electric fields for longer durations than that used in electroporation.

Sonoporation is similar to electroporation, wherein DNA is driven by an electrical force along the electric field. Sonoporation may be mediated by passive diffusion. The transfer efficiency may depend on ultrasound frequency and intensity. Low-frequency ultrasound irradiation may cause mechanical perturbation of the cell membrane, and may allow for the uptake of large molecules in the vicinity of the cavitation bubbles. The collapse of these bubbles may generate increased permeability of a cell membrane, or other delivery target or an intervening tissue, such as by the creation of small pores in a cell membrane. This, in turn, may induce a transient membrane permeabilization. This formation of small pores in a delivery target or an intervening tissue using ultrasound may allow the transfer of DNA/RNA into the cell. This phenomenon is known as sonoporation. Sonoporation uses ultrasound waves to disorganize lipids allowing the permeability of a delivery target or an intervening tissue to be increased. The presence of microbubbles may reduce the threshold of cavitation. A type of microbubble contrast agent are spheres filled with gas and stabilized with shells. One such echogenic agent, OPTISON® from GE Healthcare, Inc. (a suspension of gas-filled lipid microspheres), may be used in conjunction with the vector to enhance delivery efficacy via the larger pressures induced by microbubble-enhanced cavitation. Sonoporation may also stimulate endocytosis of AAV and enhance the efficiency of gene transfer. Alternately, hollow silica microspheres may be used instead of or in addition to lipid bubbles to enhance cavitation. One such embodiment is an amino-functionalized hollow silica microsphere with a size range of between about 1 μm and about 10 μm. Alternately, polyethylene glycol-modified liposomes containing perfluoropropane may be used as an echogenic agent to enhance cavitation. By way of nonlimiting example, ultrasound of between about 2 and about 4 MHz, between about 0.5 W/cm² and 4 W/cm², and duty cycle of between about 1% and about 10% may be applied in conjunction with a volume fraction of between about 2% to about 10% OPTISON to a target area for between about 30 seconds to about 4 minutes to improve the transdermal delivery of a therapeutic agent. Unwanted heating may occur at higher duty cycles. Cationic polymers have may also be used as carriers for gene delivery since they may condense DNA into small particles and may facilitate uptake by endocytosis. One of these cationic polymers is polyethyleneimine (PEI). PEI with a molecular weight range of between about 5 kDa and about 25 kDa may be preferred over lower-molecular-weight PEI. Lower-molecular-weight PEI may be less effective for gene delivery, due to the lower amount of positive charges per molecule that might make it difficult for such PEI configurations to appropriately condense negatively charged DNA molecules.

Laser induction (or optoporation or photoporation or laserfection or optoinjection or optical transfection or light-induced convective transmembrane transport), uses a laser pulse to transiently increase the permeability of a delivery target or an intervening tissue via a mechanism similar to that of electroporation and sonopopration, but uses optical energy to create cavitation. Current optoporation typically utilizes a short pulsed laser that may be used to create a plasma-mediated cavitation event. As described with respect to sonoporation, the collapse of these bubbles may generate increased permeability of a cell membrane, or other delivery target or an intervening tissue, such as by the creation of small pores in a cell membrane. By way of nonlimiting example, a system configured to practice the present invention may utilize a near-infrared laser to create 10 ns duration pulses that are directed to the surface of the delivery target or an intermediate tissue at an energy density of 50 J/cm², which may be required to cause dielectric breakdown and cause subsequent cavitation. Similarly, an optical scanning system may be used to direct the beam to multiple locations, such as was described above with respect to laser ablation. Of course, shorter pulse duration lasers may also be used, but the cost of such systems increases dramatically for pulse durations shorter than about 100 ps. Unlike most optical transfection applications, which are typically directed to in-vitro single cell optoporation, one embodiment of the present invention may seek to illuminate large area of skin with a single exposure in order to interrogate a plurality of delivery targets and/or intervening tissues. Such a embodiment, may be configured to utilize a Q-switched Nd:YAG laser, providing pulse durations between about ½ ns to about 100 ns and output energies of about 1/10 J to about 100 J per pulse. This may be delivered to the delivery target or intervening tissue via an optical fiber or articulating arm arrangement that ends in a handpiece to position the output of the system onto the delivery target or an intervening tissue and provide a fluence in excess of the threshold for laser-induced breakdown of water, as is described, for example, by Alfred Vogel in doi:1077-260X(96)09598-6, which is incorporated by reference herein in its entirety.

In one embodiment, microneedles contained in an array (such as the Hollow Microstructured Transdermal System® available from 3M corporation) or on a roller (such as the Dermaroller System® available from Derma Roller System Ltd) may be used to breach the stratum corneum (SC). A microneedle device may be configured such that its needles have diameters between about 50 μm and about 200 μm, and heights between about 50 μm and about 3 mm. Microneedles may be used to inject beneath the stratum corneum and into the epidermis and/or the dermis, or to perforate the SC and/or epidermis for subsequent administration of the therapeutic agent. Avoiding the highly vascularized area nearby the dermal-epidermal junction may be preferable in some instances to concentrate exposure to nervous tissue, and thus needles with lengths greater than and/or less than the epidermal thickness may be used.

Alternately, microneedles may be distributed in a nominally uniform pattern atop a 2 cm wide by 2 cm diameter cylindrical roller. As reported, the typical use of such a microneedle roller utilizing 70 μm diameter needles results in a perforation density of about 240/cm² after 10 to 15 applications over the same skin area. The therapeutic agent may be applied to the skin after perforating the SC. An occlusive dressing at least partially comprising the therapeutic agent may also be applied to the treated skin.

Alternately, a microneedle roller may be configured with the addition of central fluid chamber that is in communication with the needles. The central fluid chamber may further contain the therapeutic agent and made to dispense the therapeutic agent during a microneedling procedure. The pressure and/or flow of the agent may be further controlled to ensure its delivery.

Transdermal patches have been used for the administration of small-molecule lipophilic drugs that can be readily absorbed through the skin. In an embodiment, transdermal patches may be also used once the barrier of the stratum corneum is bypassed at least partially by, for example, one of the abovementioned processes.

The aforementioned and incorporated by reference Towne et al publication (US20160030765, or the '765 publication) describes various configurations and methods for illuminating across or through various layers of tissue, such as layers of the skin. Alternately, a more “passive” system may be employed to provide light to the target. An electrical system such as those described in the aforementioned incorporated '765 publication may be used without a substantially separate housing, and placed more directly onto the target site. These may be configured to illuminate the tissue at a specific intensity and/or duty cycle, etc. Configuration of illumination parameters may be achieved via software, as described in detail herein, or using discrete components. Such a discrete system may be made in a more cost-effect manner than a fully programmable system, and as such be more readily discarded and recycled. The low duty cycle requirements of certain opsins, such as step-function opsins, may be more amenable to such configurations due to their relatively high quantum efficiencies.

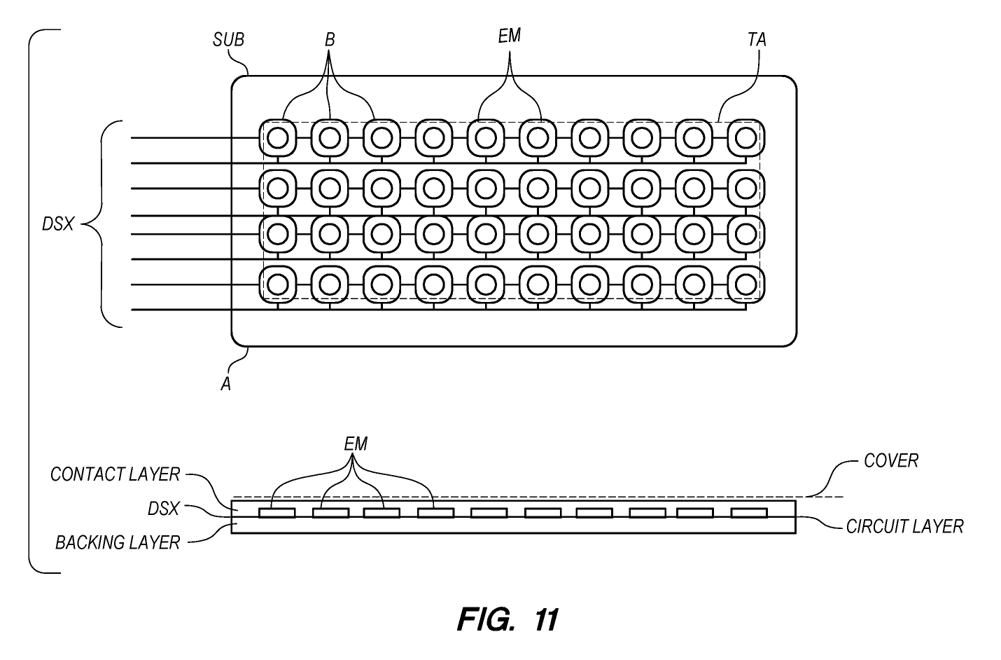

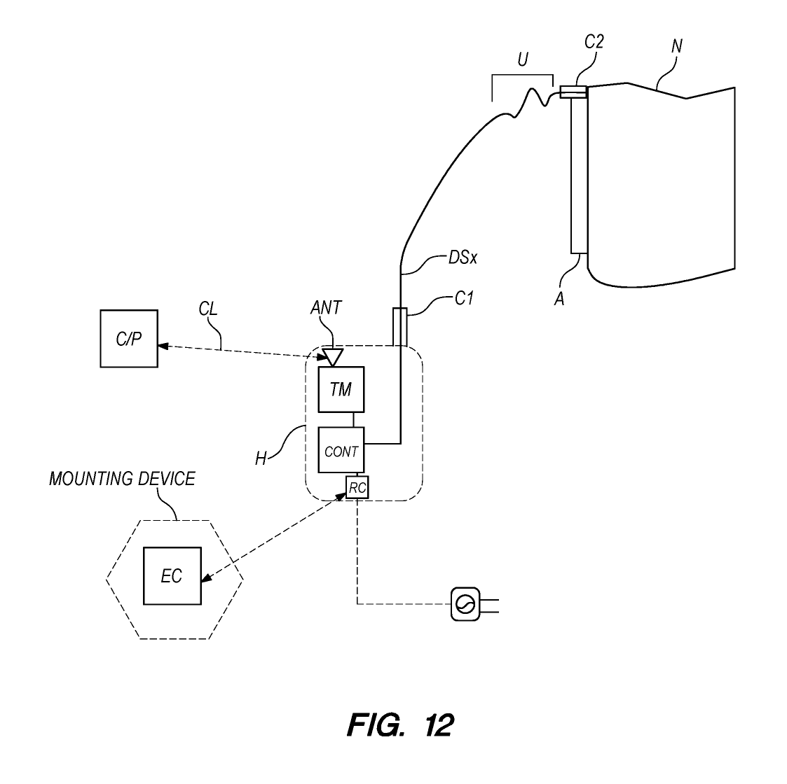

For example, such as is shown in FIG. 11 (equivalent to FIG. 135 of the '765 publication), an array of LEDs may be used to illuminate the surface of the therapeutic target, such as the skin. In this descriptive exemplary embodiment, a 2-dimensional square array of LEDs composed of emitters EM and bases B is built upon a substrate SUB, which contains a CIRCUIT LAYER with electrical current being provided by Delivery Segments DSx, a CONTACT LAYER and a BACKING LAYER. In this example rows of LEDs are arranged in a serial-parallel configuration, although other configurations are within the scope of the present invention. Emitters EM may be comprised of surface mounting LEDs, such as for example, the LUXEON Z series, or NICHIA 180A, 157X series. Emitters EM may reside on bases B in order to make electrical connections. CONTACT LAYER may be made of a nominally transparent, soft, compliant material, such as silicone, PDMS, or other such material; which may provide a level of comfort for the patient. The thickness of CONTACT LAYER may be configured to provide nominally uniform illumination at the tissue surface. For example, using LEDs from LUXEON or Cree, spaced 4 mm apart (center-to-center), illumination may be uniform to within 10% peak-to-valley using a 2.5 mm thick silicone sheet. CIRCUIT LAYER may be a single layer kapton-based flex circuit with traces configured to carry the current required that is at least in part based up on the topology, number of LEDs, and their peak powers. The number of LEDs may be chosen for a specific treatment area TA. BACKING LAYER may be constructed of a material whose compliance matches that of the CONTACT LAYER, but need not be transparent, such as Buna or other rubbers and/or polymeric materials. Both CONTACT LAYER and BACKING LAYER may be chosen to have improved thermal conductivity to limit tissue heating due to electrical inefficiencies of the LEDs, and photothermal effects due to collateral heating of tissue pigmentation. However, it should be noted that skin cooling is less of an issue for the present optogenetic therapy than for traditional laser dermatologic procedures because the irradiance used is well under those utilized for traditional laser dermatologic procedures; such as tattoo removal, vascular lesion photothermal therapy, and hair removal. These traditional therapies employ exposures of pulses from 5 ns to 500 ms and surface fluences of between 1 and 100 J/cm², which correspond to a large range of peak irradiances of between 50 mW/mm² and 20 MW/mm², albeit for short exposure times and low pulse repetition rates. Furthermore, a cover COVER may be used to keep the optical surface clean prior to use. It may alternately serve to enclose adhesive, like a bandage, for fixation to a tissue surface. Delivery segments DSx may be collected into a ribbon connector for connection to the rest of the therapeutic system, as shown in FIG. 12.

FIG. 12 (equivalent to FIG. 103 in the '765 publication) relates to an exemplary therapeutic device for use with the applicator described above with respect to FIG. 98. Applicator A, slab-type applicator that is 20 mm wide and 40 mm long, such as is described in more detail with respect to FIGS. 18 and 21-23 of International application number PCT/US2013/000262 (publication number WO/2014/081449), which is incorporated by reference herein in its entirety, is deployed about the surface of target tissue N. Electrical power is delivered to Applicator A via Delivery Segment DS to power the LEDs resident in the applicator. The resulting Light Field may be configured to provide illumination of the target tissues within the surface intensity range of 0.1-40 mW/mm², and may be dependent upon one or more of the following factors; the specific opsin used, its concentration distribution within the tissue, the tissue optical properties, and the size of the target structure(s), or its depth within a larger tissue structure. The system may be operated in a pulsed mode, where the pulse duration may be made from between 0.5 ms to 1 s, with a pulse duration of 10 ms being typically effective for inhibitory channels. Furthermore, the pulse repetition frequency (PRF) may be configured from between 0.1 Hz and 200 Hz, with a PRF of 1 Hz being typically effective for inhibitory channels. Consequently, the duty cycle ranges from 0.005% to 100%, with a duty cycle of 1% being typically effective for inhibitory channels. Although not shown for simplicity and clarity in the present figure, multiple applicators and/or delivery segments may be used for a specific target structure if it is a large target structure when compared to the optical penetration depth within that structure. Delivery Segment DS may be configured to be a ribbon cable. Delivery Segment DS may further comprise Undulations U, which may provide strain relief. Delivery Segment DS may be operatively coupled to Housing H via connector C1 and to the applicator via connector C2. The electrical power and/or current may be controlled by controller CONT, and parameters such as optical intensity, exposure time, pulse duration, pulse repetition frequency, and duty cycle may be configured. The Controller CONT shown within Housing H is a simplification, for clarity, of that described in more detail with respect to FIG. 10. External clinician programmer module and/or a patient programmer module C/P may communicate with Controller CONT via Telemetry module TM via Antenna ANT via Communications Link CL. Power Supply PS, not shown for clarity, may be wirelessly recharged using External Charger EC. Furthermore, External Charger EC may be configured to reside within a Mounting Device MOUNTING DEVICE. Mounting Device MOUNTING DEVICE may be a vest, as is especially well configured for this exemplary embodiment. External Charger EC, as well as External clinician programmer module and/or a patient programmer module C/P and Mounting Device MOUNTING DEVICE may be located within the extracorporeal space ESP, while the rest of the system is implanted and may be located within the intracorporeal space ISP. External Charger EC may also be an AC adapter, as shown by the dotted line and universal AC symbol.

A block diagram is depicted in FIG. 32 illustrating various components of an example housing H. In this example, the housing includes processor CPU, memory M, power source PS, telemetry module TM, antenna ANT, and the driving circuitry DC for an optical stimulation generator. The Housing H is shown coupled to one Delivery Segments DSx for simplicity and clarity. It may be a multi-channel device in the sense that it may be configured to include multiple electronic paths (e.g., multiple light sources and/or sensor connections) that may deliver different optical outputs, some of which may have different wavelengths. The delivery segments may be detachable from the housing, or be fixed.

Memory (MEM) may store instructions for execution by Processor CPU, optical and/or sensor data processed by sensing circuitry SC, and obtained from sensors both within the housing, such as battery level, discharge rate, etc., and those deployed outside of the Housing (H), possibly in Applicator A, such as optical and temperature sensors, and/or other information regarding therapy for the patient. Processor (CPU) may control Driving Circuitry DC to deliver power to the light source (not shown) according to a selected one or more of a plurality of programs or program groups stored in Memory (MEM). The Light Source may be internal to the housing H, or remotely located in or near the applicator (A), as previously described. Memory (MEM) may include any electronic data storage media, such as random access memory (RAM), read-only memory (ROM), electronically-erasable programmable ROM (EEPROM), flash memory, etc. Memory (MEM) may store program instructions that, when executed by Processor (CPU), cause Processor (CPU) to perform various functions ascribed to Processor (CPU) and its subsystems, such as dictate pulsing parameters for the light source, as described earlier.

In accordance with the techniques described in this disclosure, information stored in Memory (MEM) may include information regarding therapy that the patient had previously received. Storing such information may be useful for subsequent treatments such that, for example, a clinician may retrieve the stored information to determine the therapy applied to the patient during his/her last visit, in accordance with this disclosure. Processor CPU may include one or more microprocessors, digital signal processors (DSPs), application-specific integrated circuits (ASICs), field-programmable gate arrays (FPGAs), or other digital logic circuitry. Processor CPU controls operation of implantable stimulator, e.g., controls stimulation generator to deliver stimulation therapy according to a selected program or group of programs retrieved from memory (MEM). For example, processor (CPU) may control Driving Circuitry DC to deliver optical signals, e.g., as stimulation pulses, with intensities, wavelengths, pulse widths (if applicable), and rates specified by one or more stimulation programs. Processor (CPU) may also control Driving Circuitry (DC) to selectively deliver the stimulation via subsets of Delivery Segments (DSx), and with stimulation specified by one or more programs. Different delivery segments (DSx) may be directed to different target tissue sites, as was previously described.

Telemetry module (TM) may include a radio frequency (RF) transceiver to permit bi-directional communication between implantable stimulator and each of clinician programmer and patient programmer (C/P). Telemetry module (TM) may include an Antenna (ANT), of any of a variety of forms. For example, Antenna (ANT) may be formed by a conductive coil or wire embedded in a housing associated with medical device. Alternatively, antenna (ANT) may be mounted on a circuit board carrying other components of implantable stimulator or take the form of a circuit trace on the circuit board. In this way, telemetry module (TM) may permit communication with a controller/programmer (C/P). Given the energy demands and modest data-rate requirements, the Telemetry system may be configured to use inductive coupling to provide both telemetry communications and power for recharging, although a separate recharging circuit (RC) is shown in FIG. 10 for explanatory purposes.

External programming devices for patient and/or physician can be used to alter the settings and performance of the implanted housing. Similarly, the implanted apparatus may communicate with the external device to transfer information regarding system status and feedback information. This may be configured to be a PC-based system, or a stand-alone system. In either case, the system must communicate with the housing via the telemetry circuits of Telemetry Module (TM) and Antenna (ANT). Both patient and physician may utilize controller/programmers (C/P) to tailor stimulation parameters such as duration of treatment, optical intensity or amplitude, pulse width, pulse frequency, burst length, and burst rate, as is appropriate.

Once the communications link (CL) is established, data transfer between the MMN programmer/controller and the housing may begin. Examples of such data are:
1. From housing to controller/programmer:
   a. Patient usage
   b. Battery lifetime
   c. Feedback data
      i. Device diagnostics (such as direct optical transmission measurements by an emitter-opposing photosensor)
2. From controller/programmer to housing:
   d. Updated illumination level settings based upon device diagnostics
   e. Alterations to pulsing scheme
   f. Reconfiguration of embedded circuitry
      i. FPGA, etc.

By way of non-limiting examples, near field communications, either low power and/or low frequency; such as is produced by Zarlink/MicroSEMI may be employed for telemetry, as well as Bluetooth, Low Energy Bluetooth, Zigbee, etc.

Another example of a more passive system is one comprising a luminescent material instead of the electrically excited light source of FIGS. 11 and 12, but which may be contained in a similar manner to that shown for Applicator A, and not require the driving or control electronics or their connections. One nonlimiting example of such a configuration is the use of the predominantly blue light emitting chemiluminescent peroxyoxalate oxidation reactions, such as bis(2,4,6-trichlorophenyl) oxlate (TCPO)+H202. Alternately, another nonlimiting embodiment utilizes the predominantly blue light emitting chemiluminescent reaction of Luminol+H202 oxidation. Both of these chemiluminescent systems provide output in and near 450 nm, which is suitable for activating Channelrhodopsin-based opsins, such as iC++ and SwiChR. The illumination levels may be made to be on the order of 0.3 mW/mm2, and a mirrorized cover may be used to redirect light that is directed away from the target back towards it. Quenching, stabilizing, and catalyzing compounds may also be employed to prolong and stabilize light emission. One such example is the use of the following recipe for a TCPO-based solution. 15 mL of ethyl acetate, 3 mg of 9,10-bis(phenyethynyl) anthracene, 1 g of sodium acetate, and 800 mg of TCPO. This may be mixed with 3 mL of hydrogen peroxide to initiate the reaction. The design and configuration of the applicator may be substantially similar to those comprised of electrically excited light sources, such as the LEDs of FIGS. 26 & 98 in the aforementioned incorporated '765 publication to Towne et al.

Similarly, 9,10-Bis(phenylethynyl)anthracene (BPEA)and/or 2-chloro-9,10-bis(phenylethynyl)anthracene may be used instead of or in addition to the TCPO mixture described above to tailor the output spectrum of the light for use with different opsins.

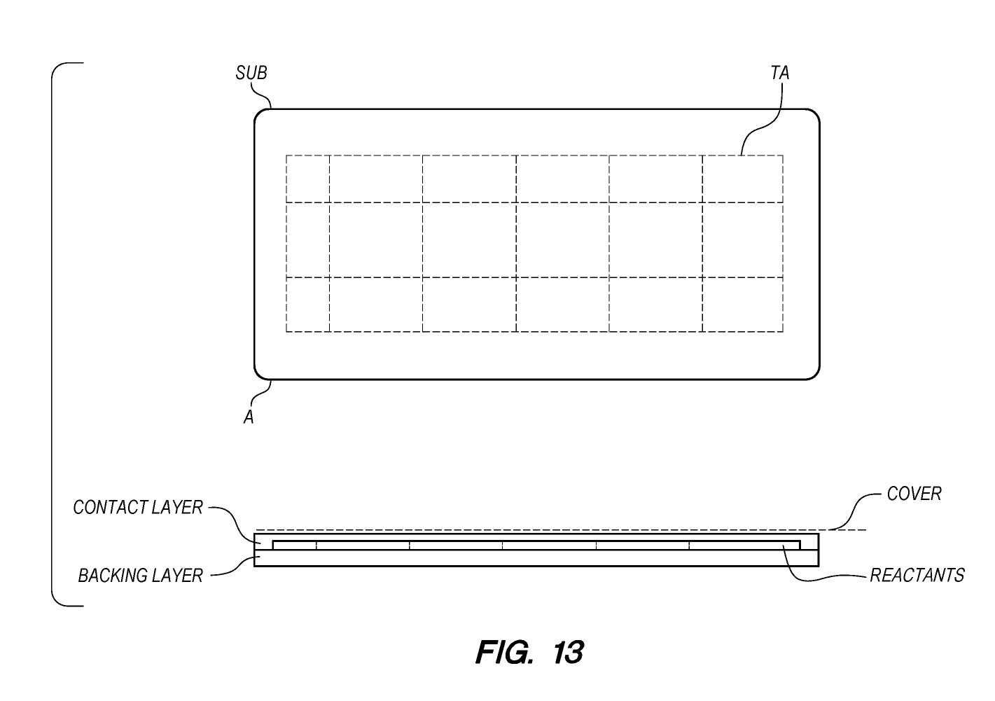

Any of these chemiluminescence-based systems may be configured to utilize a physical separation or barrier between chemical components that may be compromised prior to use, such as a brittle polymer layer in Applicator A at or around the area occupied by Circuit Layer shown in FIG. 11. An example of such a configuration is to physically separate at least one of the reaction components into a compartment, such as a silicone bag containing partial thickness perforations that tear open if stretched. FIG. 13 shows an example of such a configuration, similar to that of FIG. 11, wherein an Applicator A comprises a Reactants volume that is further configured to rupture upon mechanical stress, such as twisting or pulling and thereby release at least one of the reactants to allow mixing of the reactants to begin the chemiluminescence reaction.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element.

As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element-irrespective of whether a given number of elements are enumerated in such claims, or the addition of a feature could be ded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed:

1. A system for altering the function of a sensory unit that innervates a targeted tissue region in an animal, the sensory unit being configured to express a light-responsive protein, comprising:
a. a light delivery element configured to direct radiation to at least a portion of a targeted tissue structure; and
b. a light source configured to provide light to the light delivery element;
wherein the targeted tissue structure is illuminated transcutaneously with radiation such that a membrane potential of cells comprising the targeted tissue structure is modulated at least in part due to exposure of the light-responsive protein to the radiation.

2. The system of claim 1, wherein the light source is selected from the group consisting of a laser, a light emitting diode, and a chemiluminescent compound.

3. The system of claim 1, wherein the sensory unit is adjacent a stratum corneum layer that has been altered prior to administration of one or more clinical compounds configured to cause the sensory unit to express the light-responsive protein.

4. The system of claim 3, wherein the stratum corneum layer has been altered using a configuration selected from the group consisting of: a tape stripping configuration, a dermabrasion configuration, a microdermabrasion configuration, a depilatory compound application configuration, a sonophoresis configuration, an iontophoresis configuration, an electroporation configuration, a microdermabrasion configuration, a microneedle configuration, a laser ablation configuration, and an optoporation configuration.

5. The system of claim 1, wherein the light-responsive protein is a stimulatory opsin.

6. The system of claim 5, wherein the stimulatory opsin is selected from the group consisting of ChR2, C1V1-T, C1V1-TT, CatCh, VChR1-SFO, and ChR2-SFO.

7. The system of claim 1, wherein the light-responsive protein is an inhibitory opsin.

8. The system of claim 7, wherein the inhibitory opsin is selected from the group consisting of NpHR, eNpHR 1.0, eNpHR 2.0, eNpHR 3.0, Mac, Mac 3.0, Arch, ArchT, iChR, iC1C2, iC++, SwiChR++, and JAWS.

9. The system of claim 1, wherein the sensory unit is configured to express the light-responsive protein via administration into the targeted tissue region of an adeno-associated virus wherein a viral genome encodes at least one light responsive protein which becomes expressed in the sensory unit.

10. The system of claim 9, wherein the adeno associated virus has a coat protein selected from the group consisting of adeno-associated virus strain 1, adeno-associated virus strain 6, and adeno-associated virus strain 8.

11. The system of claim 1, wherein the targeted tissue region is selected based at least in part upon an undesired sensory response selected from the group consisting of acute pain, chronic pain, allodynia, ectopic pain, neuropathic pain, itch, and parathesia.

12. The system of claim 1, wherein the targeted tissue region is selected based at least in part upon anesthesia.

13. The system of claim 1, wherein the targeted tissue region is selected based at least in part upon a feeling of satiation.

14. The system of claim 9, wherein the adeno-associated virus is self-complementary.

15. The system of claim 2, wherein the light source is a chemiluminescent compound created using a chemiluminescent reaction that is based at least in part upon a peroxyoxalate oxidation reaction.


SEQUENCE LISTING (5 sequences)

SEQ ID NO: 1 - protein, 348 aa, Artificial sequence
  Synthetic protein
MSRRPWLLALALAVALAAGSAGASTGSDATVPVATQDGPDYVFHRAHERMLFQTSYTLENNGSVICIPNNGQCFCLAWLKSNGTNAEKLAANILQWITFALSALCLMFYGYQTWKSTCGWEEIYVATIEMIKFIIEYFHEFDEPAVIYSSNGNKTVWLRYAEWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAV

SEQ ID NO: 2 - protein, 348 aa, Artificial sequence
  Synthetic protein
MSRRPWLLALALAVALAAGSAGASTGSDATVPVATQDGPDYVFHRAHERMLFQTSYTLENNGSVICIPNNGQCFCLAWLKSNGTNAEKLAANILQWISFALSALCLMFYGYQTWKSTCGWEEIYVATISMIKFIIEYFHSFDEPAVIYSSNGNKTKWLRYASWLLTCPVILIRLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSKYGSNVGHTIIDLMSKQCWGLLGHYLRVLIHEHILIHGDIRKTTKLNIGGTEIEVETLVEDEAEAGAV

SEQ ID NO: 3 - protein, 309 aa, Artificial sequence
  Synthetic protein
MDYGGALSAVGLFQTSYTLENNGSVICIPNNGQCFCLAWLKSNGTNAEKLAANILQWISFALSALCLMFYGYQTWKSTCGWENIYVATIQMIKFIIEYFHSFDEPAVIYSSNGNKTRWLRYASWLLTCPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSRYGSNVGHTIIDLMSKQCWGLLGHYLRVLIHSHILIHGDIRKTTKLNIGGTEIEVETLVEDEAEAGAV

SEQ ID NO: 4 - protein, 309 aa, Artificial sequence
  Synthetic protein
MDYGGALSAVGLFQTSYTLENNGSVICIPNNGQCFCLAWLKSNGTNAEKLAANILQWISFALSALCLMFYGYQTWKSTCGWENIYVATIQMIKFIIEYFHSFDEPAVIYSSNGNKTRWLRYASWLLTAPVILIHLSNLTGLANDYNKRTMGLLVSDIGTIVWGTTAALSKGYVRVIFFLMGLCYGIYTFFNAAKVYIEAYHTVPKGRCRQVVTGMAWLFFVSWGMFPILFILGPEGFGVLSRYGSNVGHTIIDLMSKQCWGLLGHYLRVLIHSHILIHGDIRKTTKLNIGGTEIEVETLVEDEAEAGAV

SEQ ID NO: 5 - protein, 331 aa, Artificial sequence
  Synthetic protein
MASQVVYGEWASTHTECYNMSRIDSTFVSLLQLVWAVVSGCQTIFIMSRAPKVPWESVYLPFVESITYALASTGNGTLQMRDGRFFPWSRMASWLCTCPIMLGQISNMALVKYKSIPLNPIAQAASIIRVVMGITATISPAEYMKWLFFFFGATCLVFEYSVVFTIFQVGLYGFESVGTPLAQKVVVRIKMLRLIFFIAWTMFPIVWLISPTGVCVIHENVSAILYLLADGLCKNTYGVILWSTAWGVLEGKWDPPACLPGQEKPEADDPFGLNHEKNAPPNDEVNIRMFGRVIGSVRKSKRRQKWELAPAHLEDRIRLSDEESDDSRPKR